(19) United States
(12) Patent Application Publication
(10) Pub. No.: US 2022/0160688 A1
(43) Pub. Date: May 26, 2022

(54) INHIBITION OF NAMPT AND/OR SARM1 FOR THE TREATMENT OF AXONAL DEGRADATION

(71) Applicant: THE JOHNS HOPKINS UNIVERSITY, Baltimore, MD (US)

(72) Inventors: Vassilis E. KOLIATSOS, Baltimore, MD (US); Nikolaos ZIOGAS, Baltimore, MD (US); Athanasios ALEXANDRIS, Baltimore, MD (US); Jiwon RYU, Baltimore, MD (US)

(21) Appl. No.: 17/441,149

(22) PCT Filed: Mar. 20, 2020
(86) PCT No.: PCT/US2020/023755

§ 371 Date: Sep. 20, 2021

Related U.S. Application Data
(63) Continuation of application No. PCT/US2020/021155, filed on Mar. 5, 2020.
(60) Provisional application No. 62/821,214, filed on Mar. 20, 2019.

(51) Int. Cl. A61K 31/444; A61K 31/4409; A61K 31/4439; A61K 31/4406; A61P 25/28; A61P 25/02

(52) U.S. Cl. CPC A61K 31/444; A61K 31/4409; A61P 25/02; A61K 31/4406; A61P 25/28; A61K 31/4439

(57) ABSTRACT

This invention relates generally to diseases and conditions characterized with axonal degradation and, more particularly, to methods and compositions for treating or preventing traumatic or degenerative neuropathies and other diseases and conditions involving axonal breakdown/degeneration.

### FEDERALLY SPONSORED RESEARCH OR DEVELOPMENT

This invention was made with government support under grant number EY028039 awarded by the National Institutes of Health. The government has certain rights in the invention

### FIELD OF THE INVENTION

This invention relates generally to diseases and conditions characterized with axonal degradation and, more particularly, to methods and compositions for treating or preventing traumatic or degenerative neuropathies and other diseases and conditions involving axonal breakdown/degeneration.

### BACKGROUND OF THE INVENTION

Cells undergo regulated self-destruction during development and in response to stresses (see, Fuchs Y, Steller H. Cell. 2011; 147:742-758). Axons, the longest cellular structures in the body that also contain the majority of neuronal cytoplasm, have a locally mediated self-destruction program that promotes axonal breakdown and removes damaged axons in the setting of neurological disorders (see, Conforti L, et al., Nat. Rev. Neurosci. 2014; 15:394-409).

Axonal degeneration occurs in the course of traumatic, toxic, metabolic or ischemic injury and in a variety of neurodegenerative diseases such as Parkinson's, Alzheimer's diseases and Amyotrophic Lateral Sclerosis. Such diseases and conditions are associated with functional as well as structural demise of axons. The paradigmatic form of axonopathy is Wallerian degeneration, which occurs when the distal portion of the axon is severed from the cell body. The severed axon rapidly succumbs to a strictly regulated, orderly, complete degeneration. Although, in the peripheral nervous system (PNS), the apparent function of such a programmatic axonal breakdown is to clear dysfunctional axons and prepare the ground for regeneration, an ongoing chronic activation of such programs may override the regenerative potential of the nerve. In the central nervous system (CNS), repair/regeneration does not occur or is incomplete. Wallerian degeneration and related processes lead to a loss of large numbers of axons, disconnection among brain regions, brain atrophy, and chronic disability. Because there are no known techniques for intervening to repair axons once degeneration sets in, especially in the CNS, the most effective strategy is to prevent or promptly mitigate Wallerian-type and related axonal breakdown programs in the initiation phase.

Axonal degeneration is a hallmark of peripheral neuropathy, glaucoma, brain injury (e.g., traumatic brain injury), and neurodegenerative disease. Neurodegeneration and neurodegenerative disorders include progressive dysfunction and/or loss of nerve or glial cells in the PNS and CNS. In all these conditions, as explained above, axonopathy is a critical feature of pathology responsible for disease progression, symptoms and signs, and accounts for a large portion of a patient's disability.

Improved methods for treating axon degradation and related processes are needed.

The present invention addresses this need.

### SUMMARY

Traumatic brain injury (TBI) is associated with mixed neuropathologies, including contusions, diffuse or traumatic axonal injury (TAD, meningeal or parenchymal hemorrhage, and protein aggregation. TAI is the most common pathology regardless of TBI cause or severity and is thought to be the result of dynamic loading of axons during rotational acceleration of the head (see, Maxwell W L, et al., (1993) Acta Neuropathol 86:136-144; Smith D H, et al., (1997) J. Neuropathol Exp Neurol 56: 822-834; Smith D H, et al., (2003) J. Head Trauma Rehabil 18:307-316). This mechanism causes immediate axonal disruption leading to secondary axonal or perikaryal degeneration. One of the distinguishing features of TAI is that injury begins at the axon, as contrasted to axonal damage or degeneration that is secondary to perikaryal injury or cell death.

As noted, TAI is a common neuropathology in traumatic brain injury and is featured by primary injury to axons. Experiments conducted during the course of developing embodiments for the present invention generated TAI with impact acceleration of the head in male Thy1-eYFP-H transgenic mice in which specific populations of neurons and their axons were labeled with yellow fluorescent protein. This model resulted in axonal lesions in multiple axonal tracts along with blood-brain barrier disruption and neuroinflammation, that is neuropathologies typical of TBI but also many other diseases of the nervous system. The corticospinal tract, a prototypical long tract, is severely affected. Using optimized CLARITY at single-axon resolution in such experiments, the entire corticospinal tract volume from the pons to the cervical spinal cord was visualized in 3D and the total number of axonal lesions and their progression over time counted. These assessments divulged the presence of progressive traumatic axonopathy that was maximal at the pyramidal decussation. The perikarya of injured corticospinal neurons atrophied, but there was no evidence of neuronal cell death. CLARITY was also used at single-axon resolution to explore the role of the nicotinamide mononucleotide adenyltransferase 2 (NMNAT2)/sterile alpha toll-interleukin receptor motif containing protein (SARM1) axonal self-destruction pathway in traumatic axonopathy. The protein SARM1 is an essential mediator of axon degeneration. SARM1 is a negative regulator of Toll-Like Receptor-activated transcriptional programs, but its mechanism for axon degeneration is unknown. Additional experiments demonstrated that inhibition of NAMPT, the rate limiting enzyme in the salvage NAD+ biosynthetic pathway, also offered protection against Wallerian degeneration.

Experiments were conducted that interfered with the NMNAT2/SARM1 axonal destruction pathway by genetic ablation of SARM1 or by pharmacological strategies designed to block NAMPT, thereby decreasing levels of nicotinamide mononucleotide (NMN), an NMNAT2 substrate that may have adverse properties under conditions of injury and may also increase levels of Nicotinamide (Nam). Both strategies may interfere with SARM1 activity, although mechanisms are still unknown. These interventions resulted in a significant reduction in the number of axonal lesions early after injury, both in vitro and in vivo, and anti-SARM1 interventions offered long-term anatomical and functional/behavioral benefits.

In the absence of available SARM1 inhibitors, experiments described herein established the inhibition of the NMN synthesizing enzyme NAMPT, as an alternative therapeutic strategy in axotomy models in vitro and in vivo. A more detailed analysis was undertaken in vitro. Wild type or SARM1 KO embryonic mouse dorsal root ganglion (DRG) cells were cultured in poly-dimethylsiloxane microfluidic devises for axon compartmentalization. Axons were cut with a razor blade and treated with the NAMPT inhibitor FK866 or vehicle at different time points. Images of axons were taken at different intervals and the degeneration index was calculated. Maximum protection against WD was observed with treatment at 2 hours post-injury which was similar to the protection afforded by SARM1 deletion. Importantly treatment after the onset of visible fragmentation was still effective in delaying further fragmentation. A group of other available NAMPT inhibitors (CHS-828, GPP78, STF118804, STF31) was also tested at different concentrations and showed comparably strong efficacy. Axonal preparations at different time points following axotomy were also used for metabolomic profiling and western blot assessment of injury related signals. Such experiments indicated that NAMPT inhibition by a variety of small molecules—some of which have already been tested in clinical trials—is an attractive therapeutic strategy against WD. Most importantly such experiments demonstrated that the molecular decision for WD is stochastic and temporally separated from the time of injury, effectively allowing for a clinically relevant therapeutic window of intervention.

The above findings demonstrate that high-resolution strategies in vitro and in vivo optimized to serve as neuropathological tools reveal important features of TAI with biological implications, especially the progressive axonopathic nature of TAI and the role of the NMNAT2-SARM1 and NAMPT signaling in the early stages of axonopathy.

Such experiments further demonstrated that treatment of TAI through inhibition of SARM1 activity and/or expression and/or inhibition of NAMPT activity and/or expression is optimal with a therapeutic window of treatment onset within approximately two to six hours of injury (e.g., 3-5 hours, 2-5 hours, 2-7 hours, 2.5 hours to 6.5 hours, 1.5 to 6.5 hours, 1 to 7 hours, etc.), and that treatment should last at least approximately three days after injury (e.g., 2.5 days, 2.8 days, 3.1 days, 3.5 days, 4 days, 5 days, 7 days, 10 days, 14 days, 21 days, 100 days, etc).

Accordingly, the present inventors have succeeded in discovering that axonal degeneration can be diminished or prevented by interfering with the NMNAT2/SARM1 and/or NAMPT signaling of axonal destruction in diseased and/or injured neurons through blocking SARM1 activity and/or expression and/or inhibiting NAMPT activity and/or expression in such diseased and/or injured neurons. Thus, one approach to preventing axonal degeneration can be by interfering with the NMNAT2/SARM1 and/or NAMPT signaling of axonal destruction in injured mammalian axons through inhibiting SARM1 activity and/or expression and/or NAMPT activity and/or expression in such injured mammalian axons. The inhibition of SARM1 can be through direct action on SARM1 or by increasing the supply of Nam (e.g., through administration of FK866) which may act a feedback inhibitor of SARM1. The inhibition of NAMPT can be through direct action on NAMPT (e.g., through administration of NAMPT inhibitors (e.g., FK866, CHS-828, GPP78, STF118804, STF31) (e.g., a chemically related or unrelated molecule that inhibits NAMPT) and may have effects in addition to increasing Nam and presumably inhibiting SARM1. The inhibition of SARM1 and/or NAMPT results in a decrease in severity of axonal degeneration or prevention of axonal degeneration. Such treatment is optimized when administered within two to six hours of injury (e.g., 3-5 hours, 2-5 hours, 2-7 hours, 2.5 hours to 6.5 hours, 1.5 to 6.5 hours, 1 to 7 hours, etc.), and for a duration between three to fourteen days in vivo (e.g., in the case of a chronic neurodegenerative disease, such a treatment can be delivered in repeat epochs lasting at least 3 days each) (e.g., 2.5 days, 2.8 days, 3.1 days, 3.5 days, 4 days, 5 days, 7 days, 10 days, 14 days, 21 days, 100 days, etc).

Thus, in certain embodiments, the present invention is directed to a method of treating or preventing a disease or condition characterized by axonal degradation or neuropathy characterized with axonal degradation in a mammal and, in particular, in a human in need thereof.

Such methods can comprise administering an effective amount of an agent that acts to interfere with the NMNAT2/SARM1 signaling of axonal destruction in diseased and/or injured neurons through inhibiting SARM1 activity and/or expression in such diseased and/or injured neurons.

Such methods can comprise administering an effective amount of an agent that acts to interfere with NAMPT signaling of axonal destruction in diseased and/or injured neurons through inhibiting NAMPT activity and/or expression in such diseased and/or injured neurons.

Such methods can comprise administering an effective amount of an agent that acts to interfere with NMNAT2/SARM1 and NAMPT signaling of axonal destruction in diseased and/or injured neurons through inhibiting SARM1 and NAMPT activity and/or expression in such diseased and/or injured neurons.

In certain embodiments, the present invention provides methods for reducing axonal degradation in a neuron. In some embodiments, these methods are performed in vitro. In other embodiments, the methods are performed in vivo.

Such methods can include selecting, providing, or obtaining a neuron with, undergoing, or at risk for axonal degradation, and contacting or treating the neuron with an effective amount of a composition that interferes with the NMNAT2/SARM1 signaling of axonal destruction pathway in such diseased and/or injured neurons through inhibiting SARM1 activity and/or expression for a time sufficient to inhibit SARM1 activity and/or expression, thereby reducing axonal degradation in the neuron.

Such methods can include selecting, providing, or obtaining a neuron with, undergoing, or at risk for axonal degradation, and contacting or treating the neuron with an effective amount of a composition that interferes with the NAMPT signaling of axonal destruction in such diseased and/or injured neurons through inhibiting NAMPT activity and/or expression for a time sufficient to inhibit NAMPT activity and/or expression, thereby reducing axonal degradation in the neuron.

Such methods can include selecting, providing, or obtaining a neuron with, undergoing, or at risk for axonal degradation, and contacting or treating the neuron with an effective amount of a composition that interferes with NMNAT2/SARM1 and NAMPT signaling of axonal destruction in such diseased and/or injured neurons through inhibiting SARM1 and NAMPT activity and/or expression for a time sufficient to inhibit SARM1 and NAMPT activity and/or expression, thereby reducing axonal degradation in the neuron.

In certain embodiments, the present invention provides methods for reducing axonal degradation in a subject with or at risk for developing axonal degradation, for example, in the central and/or peripheral nervous system. Such treatment is optimized when administered within two to six hours of injury (e.g., 3-5 hours, 2-5 hours, 2-7 hours, 2.5 hours to 6.5 hours, 1.5 to 6.5 hours, 1 to 7 hours, etc.), and for a duration between three to fourteen days in vivo (e.g., in the case of a chronic neurodegenerative disease, such a treatment can be delivered in repeat epochs lasting at least 3 days each) (e.g., 2.5 days, 2.8 days, 3.1 days, 3.5 days, 4 days, 5 days, 7 days, 10 days, 14 days, 21 days, 100 days, etc).

Such methods can include selecting a subject with or at risk for developing axonal degradation, and treating the subject with, or administering to the subject, an effective amount or dose of a composition that interferes with NMNAT2/SARM1 signaling of axonal destruction in such diseased and/or injured neurons through inhibiting SARM1 activity, thereby reducing axonal degradation in the subject.

Such methods can include selecting a subject with or at risk for developing axonal degradation, and treating the subject with, or administering to the subject, an effective amount or dose of a composition that interferes with NAMPT axonal destruction pathway in such diseased and/or injured neurons through inhibiting NAMPT activity, thereby reducing axonal degradation in the subject.

Such methods can include selecting a subject with or at risk for developing axonal degradation, and treating the subject with, or administering to the subject, an effective amount or dose of a composition that interferes with NMNAT2/SARM1 and NAMPT signaling of axonal destruction in such diseased and/or injured neurons through inhibiting SARM1 and NAMPT activity, thereby reducing axonal degradation in the subject.

In some embodiments, subjects suitable for treatment can have or be at risk for suffering a traumatic brain injury. In some embodiments, subjects suitable for treatment can have or be at risk of developing neurodegenerative disease. In addition, such subjects can have or be at risk of developing axonal degradation is in the central and/or peripheral nervous system. In some embodiments, a subject with or at risk of developing axonal degradation can have diabetes and/or diabetic neuropathy and/or can undergo chemotherapy and have chemotherapy-induced peripheral neuropathy (e.g., peripheral neuropathy) and/or have glaucoma.

Such methods are not limited to an agent or manner of decreasing or inhibiting SARM1 activity and/or expression. In some embodiments, the agent can decrease SARM1 activity through increasing nicotinamide (Nam) levels (as nicotinamide may serve as a feedback inhibitor of SARM1 activity). Such agents can include any agent that increases Nam levels in relevant cells (e.g., diseased and/or injured neurons), increases a precursor of Nam in relevant cells, an intermediate of Nam in relevant cells, and/or an intermediate in a pathway resulting in increase of Nam levels in relevant cells. In some embodiments, the agent (e.g., the agent that increases Nam levels in relevant cells) is FK866. In some embodiments, the agent is any type of small molecule compound or pharmaceutical formulation that is capable of decreasing SARM1 activity and/or expression. In various embodiments, the agent decreases SARM1 activity through inhibiting SARM1 genetic expression (e.g., gene therapy) (e.g., genetic ablation of SARM1 expression).

Such methods are not limited to an agent or manner of decreasing or inhibiting NAMPT activity and/or expression. In some embodiments, the agent is a NAMPT inhibitor. In some embodiments, the NAMPT inhibitor is selected from FK866, CHS-828, GPP78, STF118804, and STF31. In some embodiments, the agent is any type of small molecule compound or pharmaceutical formulation that is capable of inhibiting NAMPT activity and/or expression. In various embodiments, the agent decreases NAMPT activity through inhibiting NAMPT genetic expression (e.g., gene therapy) (e.g., genetic ablation of NAMPT expression).

Such methods are not limited to treating a particular disease or condition characterized with axonal degradation. In some embodiments, the disease or condition characterized with axonal degradation includes, but is not limited to, axonal degradation conditions that are hereditary or congenital or associated with traumatic brain injury, or mixed traumatic neuropathologies (e.g., contusions, diffuse or traumatic axonal injury (TAD, meningeal or parenchymal hemorrhage, and protein aggregation), or with Parkinson's disease, Alzheimer's disease, Amyotrophic Lateral Sclerosis, Herpes infection, diabetes, amyotrophic lateral sclerosis, a demyelinating disease, ischemia or stroke, chemical injury, thermal injury, and AIDS.

Such methods are not limited to a particular time for onset of treatment and/or treatment duration. In some embodiments involving the treatment of traumatic brain injury or mixed traumatic neuropathologies (e.g., contusions, diffuse or traumatic axonal injury (TAI), meningeal or parenchymal hemorrhage, and protein aggregation), the treatment is administered within approximately two to six hours of injury onset (e.g., 3-5 hours, 2-5 hours, 2-7 hours, 2.5 hours to 6.5 hours, 1.5 to 6.5 hours, 1 to 7 hours, etc.), and treatment duration is between approximately three to fourteen days (e.g., 2.5 days, 2.8 days, 3.1 days, 3.5 days, 4 days, 5 days, 7 days, 10 days, 14 days, 21 days, 100 days, etc). In some embodiments, such treatments may involve repeat administrations lasting at least 3 days each.

In certain embodiments, the present invention is also directed to methods of screening agents for treating a neuropathy in a mammal. The methods can comprise administering to neuronal cells in vitro or in vivo, a candidate agent, producing an axonal injury to the neuronal cells and detecting a decrease in axonal degeneration of the injured neuronal cells. In various embodiments, the method can comprise detecting SARM1 activity and/or expression produced by a candidate agent, in a cell and, in particular, in a neuronal cell. In various embodiments, the method can comprise detecting NAMPT activity and/or expression produced by a candidate agent, in a cell and, in particular, in a neuronal cell. In various embodiments, the method can comprise detecting SARM1 and NAMPT activity and/or expression produced by a candidate agent, in a cell and, in particular, in a neuronal cell.

Methods are also provided for screening agents that decrease SARM1 activity in neurons as well as for screening agents that increase Nam levels in neurons. The methods can comprise administering to mammalian neuronal cells in vitro or in vivo a candidate agent, producing an axonal injury to the neuronal cells and detecting a decrease in axonal degeneration of the injured neuronal cells. Such methods can in some embodiments be primary screening methods in which secondary assays further delineate activity as associated with SARM1 activity or with Nam and enzymes or components of Nam biosynthetic or salvage pathways.

Methods are also provided for screening agents that decrease NAMPT activity in neurons. The methods can comprise administering to mammalian neuronal cells in vitro or in vivo a candidate agent, producing an axonal injury to the neuronal cells and detecting a decrease in axonal degeneration of the injured neuronal cells.

Methods are also provided for screening agents that decrease SARM1 and NAMPT activity in neurons. The methods can comprise administering to mammalian neuronal cells in vitro or in vivo a candidate agent, producing an axonal injury to the neuronal cells and detecting a decrease in axonal degeneration of the injured neuronal cells.

In various embodiments of the screening methods of the present invention, axonal injury can be produced by a number of methods including chemically injuring the neuronal cells, thermally injuring the neuronal cells, oxygen-depriving the neuronal cells, and physically injuring the neuronal cells.

In certain embodiments, the present invention provides compositions comprising 1) an agent that acts by decreasing SARM1 and/or NAMPT activity and/or expression in diseased and/or injured neurons and supporting cells, and 2) nicotinamide riboside (NAR).

In some embodiments, the agent inhibiting NAMPT activity is selected from FK866, CHS-828, GPP78, STF118804, STF31, and a chemically related or unrelated molecule that inhibits NAMPT.

In certain embodiments, the present invention provides methods of treating or preventing a neuropathy or axonopathy in a mammal in need thereof, the method comprising administering to the mammal an effective amount of such a composition comprising 1) an agent that acts by decreasing SARM1 and/or NAMPT activity and/or expression in diseased and/or injured neurons and supporting cells, and 2) nicotinamide riboside (NAR). In some embodiments, the neuropathy or axonopathy is hereditary or congenital or associated with neurodegenerative disease, motor neuron disease, neoplasia, endocrine disorder, metabolic disease, nutritional deficiency, atherosclerosis, an autoimmune disease, mechanical injury, chemical or drug-induced injury, thermal injury, radiation injury, nerve compression, retinal or optic nerve disorder, mitochondrial dysfunction, progressive dementia demyelinating diseases ischemia and/or stroke infectious disease; or inflammatory disease. In some embodiments, the neuropathy or axonopathy is caused by a traumatic brain injury, wherein the onset of treating is within one to seven hours of injury, wherein the duration of treating is between three to fourteen days. In some embodiments, the mammal is a human. In some embodiments, the onset of treating is within 3-5 hours, 2-5 hours, 2-6 hours, 2-7 hours, 2.5 hours to 6.5 hours, or 1.5 to 6.5 hours.

### BRIEF DESCRIPTION OF THE DRAWINGS

FIG. 1: Main features of the IA model. A, These stills from a high-speed video camera monitoring (20,000 frames per second) of an IA session depict two key events in the IA sequence, including a push-down phase with both translational and rotational acceleration, and then an upward whiplash phase primarily involving rotational acceleration of the head. Main rotational events are depicted with geometrical shapes on the right (red arrows), next to the corresponding halftones. B, Prussian blue staining at 7 d after injury shows the lack of hemosiderin signal in mouse cortex under the disc (impact site), a finding consistent with absence of contusion. Inset, Positive diffuse low signal around the mesencephalic flexure of the brainstem, site that may correspond to the concentration of acceleration or other injurious events. Scale bar, 100 μm. C, This H&E-stained section through the CA1 sector of hippocampus at 7 d after injury shows lack of eosinophilia in pyramidal neurons, suggestive of absence of anoxic-ischemic injury. Scale bar, 30 μm.

FIG. 2: Multifocal traumatic axonopathy resulting from IA of the head in YFP-H transgenic mice. Only representative fiber tracts are shown. A, B, A sagittal section through the pyramids (A) and a transverse section through the dorsal cervical cord (B) show characteristic undulations, swellings, and bulbs consistent with traumatic damage to axons (arrows). Scale bars: A, 30 μm; B, 50 μm. C, *Bulbs in the large diameter axons of the RST. Bottom left, A classical Wallerian degeneration profile (arrows). Scale bar, 20 μm. D, Numerous large swellings near the termination of the gracile fasciculus in the gracile nucleus. Scale bar, 50 μm. E, F, Small swellings, only seen in injured subjects, are distributed along the precommissural fornix (E, arrows) and near the termination of postcommissural fornix at the mammillary bodies (F, arrows). Scale bar, 50 μm. Insets, Images from sham controls for each axonal pathway.

FIG. 3: BBB disruption in brainstem shortly after IA injury. A, This preparation labeled with LEA for capillary endothelial cells and IgG for the presence of plasma in brain parenchyma shows a group of capillaries (green) perpendicular to the mesencephalic junction (*, bottom) and IgG leaking out next to vessel walls (red) 15 min after injury. Scale bar, 50 μm. B-D, This series of images from subjects perfused with LEA and processed 15 min after injury illustrates the precise extra-endothelial localization of IgG (B, D, arrows). B, Capillary is cut transversely. D, Capillary is cut longitudinally. C, Case illustrated is from a sham-injured mouse. Scale bar, 10 μm, e, Endothelial cell nucleus.

FIG. 4: Neuroinflammatory responses in YFP mice after IA. A-C, Resident microglial activation in select portions of the CST, including decussation of the pyramids (A) and the crossing of the lower pyramids with the RST (B, C) at 48 h after injury. *Main areas of activation. A, B, Sections immunolabeled with antibodies against CD68. C, Section immunostained with an antibody against P2Y12. C, Insets, Representative transformed P2Y12 (+) profiles from the same region. Top, Inset, Two clusters of such cells. Bottom inset, Two characteristic macrophages with stout, thorny processes. Scale bars: A, B, 50 μm; C, 70 μm. D-F, Blood-borne parenchymal (D, E) and pial (F) inflammatory responses at 48 h after injury. D, E, Sections immunostained with the peripheral macrophage marker F4/80. F, Section immunolabeled with antibody CD68. D, F, YFP-labeled injured axons. All images are from the lower pyramids. D, E, Pericapillary “rings” of blood-borne macrophages; these are indicated with an asterisk in D and further magnified in inset (inset counterstained with DAPI to show the presence of a capillary). E, Arrows indicate macrophages. F, Arrows indicate pial CD68 (+) macrophages. Scale bars: D, 30 μm; E, F, 5 μm. G, In this CD68-immunolabeled section, there is a cluster of transformed CD68 (+) microglial cells surrounding the end bulb of an injured axon. Such profiles can be occasionally observed 48 h after IA. Scale bar, 60 μm.

FIG. 5: Further characterization of traumatic axonopathy in the CST with confocal microscopy and axon lesion counts. A, B, Confocal images of sham (A) and injured (B) CST: one with conventional confocal microscopy (B) and another using edge-detection function (B′), highlighting classical spindle-like axonal swellings (i.e., the predominant lesion in the CST 3 h after injury). *Presence of an axon bulb. The edge detection function better illustrates the fact that a single axon can have multiple varicosities (axon bridges between them are indicated with arrows), as well as the accumulation of organelles in such swellings, indicating impaired axonal transport. Inset, *Magnification of swelling. Scale bar, 5 μm. C, Severity of axonopathy corresponds to severity of impact. Here, axonal swellings (examples are circled in bottom left) were counted in standard cross-sections of pyramids at mid-medullary level. Sham-injured mice have very few swellings (top left). Number of lesions increase with weight burden. Bars here represent mean±SEM. Group values were analyzed with one-way ANOVA followed by Tukey's post hoc testing: *p<0.05; **p<0.001. Scale bar, 40 μm.

FIG. 6: Ultrastructural profiles of damaged corticospinal axons 24 h after IA, including changes in the axoplasm (A-F) and the myelin sheath (G, Key axons are labeled with “a” everywhere. A, Differentiation between normal corticospinal axons (e.g., a₁, left top) and damaged axons (e.g., a₂, center bottom), in the early stages of axonopathy. Scale bar, 1 μm. B, Axoplasmic features of damaged axons in early stages. Note internalized myelin profiles (min) and accumulations of swollen mitochondria (mt). Scale bar, 500 nm. C, Swollen mitochondria as in B shown here at higher magnification, along with dark inclusions that are characteristic of traumatic axonopathy in relatively early stages. Inset, Normal mitochondria of an uninjured axon are shown for comparison. Scale bar, 50 nm. D, Here we show a paranodal region (*oligodendrocyte cytoplasm), with accumulations of abnormal organelles and dark inclusions. Scale bar, 500 nm. E, F, Degenerating axoplasmic features. Note a breakdown of axoplasmic structure, with the formation of vacuoles of various sizes, from relatively small (E) to large (F) (*). Scale bar, 500 nm. G, H, Abnormal myelin profiles in advanced degeneration (arrows). G, Note the difference between a normal appearing (a₁) and degenerating (a₂) axons. Scale bar, 1 μm. I, A characteristic macrophage (M) with dark inclusions (arrows) in the CST. Scale bar, 1 μm.

FIG. 7: Retrograde changes in axotomized corticospinal neurons include c-Jun phosphorylation and perikaryal atrophy. A, Injection of the retrograde label CTB in the ponto-medullary junction (inset) leads to robust perikaryal labeling of corticospinal layer V pyramidal neurons. Scale bar, 80 μm. B, C, Axonal injury is associated with phosphorylation of c-Jun in the nucleus of CST layer V pyramidal neurons (B, C, arrows). There is perikaryal atrophy in double-labeled neurons compared with neurons that do not express p-c-Jun. B, Inset, Confocal image of a double-labeled neuron. Scale bar, 50 μm. D, Phosphorylated c-Jun expression (shown here 3 d after IA) is confined to layer V and is specific to injured animals (bottom). Scale bar, 100 μm. E, These three panels show the course of retrograde changes in identified corticospinal pyramidal neurons at 3, 7, and 14 d after injury based on perikaryal volume (left), p-c-Jun expression (right), and comparison in perikaryal volume between p-c-Jun (+) and p-c-Jun (−) neurons (middle; reflecting the relationship between atrophy and p-c-Jun immunoreactivity). Error bars indicate mean±SEM. Group values were analyzed with one-way ANOVA followed by Tukey's post hoc testing for multiple comparisons or Student's t test for single comparison analysis: *p<0.05; **p<0.01; ***p<0.001.

FIG. 8: A representative TUNEL preparation through motor cortex shows the absence of cell death of cortical neurons after IA injury. This section is taken from an injured mouse 30 d after injury. Inset, Positive control section from an uninjured mouse after treatment with DNase I. Scale bar, 50 μm.

FIG. 9: Anatomical features of traumatic axonopathy in the brainstem based on CLARITY. A, A transparent oblique 3D view of the injured brainstem, including the CST, the RST, and the gracile fasciculus (FGr) at 3 h after injury. Pathology in the CST is concentrated in regions where the tract crosses the ventrally curving reticulospinal fibers and also crosses the midline and assumes a dorsal position in the upper cervical cord (dCST). Scale bar, 750 μm. B, C, Further detail of two segments of the CST using digital slices of A: one at the pons with few axonal lesions (B), and the other next to the crossing with reticulospinal fibers, with many more lesions (C). Scale bar, 80 μm. D, E, These digital slices of A show characteristic lesions in tracts related to CST in the lower brainstem, including a Wallerian profile in a reticulospinal axon (D) and the characteristic large bulbous swellings in the gracile fasciculus (E). Scale bars: D, 15 μm; B, 50 μm.

FIG. 10: Quantitative analysis of CST lesions based on CLARITY. A, B, A sagittal image of a cleared brainstem focusing on the CST and showing the concentration of axonal lesions toward the CST×RST junction and decussation (A). The CST×RST junction in (A) is further enlarged in the bottom left panel (B) and apposed to a sham brainstem shown on top. C, A computerized graphic of lesion density, based on the spot detection function. D, Progressive magnification of the 3D dataset showing the clear distinction of individual axonal lesions in the native resolution of the image acquisition. Scale bars: A, 150 μm; B, 180 μm; D (starting from top), 150 μm, 70 μm, 10 μm, respectively. E, Counts of total numbers of axonal lesions based on time (3 and 24 h after injury) (left), and on location, such as crossing with reticulospinal axons and the decussation at 3 (middle) and 24 (right) hours after injury. Error bars indicate mean±SEM. Group values were analyzed with one-way ANOVA followed by Tukey's post hoc testing: *p<0.05; **p<0.001.

FIG. 11: Comparison between YFP-based and APP-labeled axonal lesions with CLARITY at 3 and 24 h after injury. A, A sagittal view of a cleared brainstem preparation that was also processed for APP IHC (red), visualized here with 2-photon microscopy. For YFP excitation, the 2-photon laser was tuned at 920 nm and for the APP at 780 nm. Scale bar, 300 μm. B, C, Two digital slices from the 3D dataset represent magnifications of boxed areas in A, allowing the visualization of all YFP-filled and APP-labeled axonal lesions at the pyramidal decussation (B) and the crossing of the CST with the RST (C). Panels represent YFP-filled axonal lesions only (green), APP-labeled axonal lesions only (red), or both (red over green). Scale bars: B, 150 μm; C, 200 μm. D, Counts of total numbers of APP-immunoreactive abnormalities at 3 and 24 h after injury (left), densities of APP-positive abnormalities at 3 or 24 h (middle), and rates of colocalization of YFP with APP lesions at the two time points depicted here. Only a subset of YFP-filled axons immunoreact with APP. This is shown by both total counts (compare with counts of FIG. 10E) and percentages shown on the right. Error bars indicate mean±SEM. Group values were analyzed with one-way ANOVA followed by Tukey's post hoc testing (first three graphs) or Student's t test (fourth graph): *p<0.05; **p<0.01; ***p<0.001.

FIG. 12: Protective effect of SARM1 deletion on axonal pathology in CST 24 h after injury. A, Diagrams of simplified NMNAT2-SARM1 reactions relevant to FIG. 12 and FIG. 13. In the normal condition (first graph), nicotinamide (Nam) is converted to NMN and then NAD⁺ via the actions of NAMPT and NMNAT2; in this scenario, SARM1 is inactivated. In the injury condition (second graph), NMNAT2 is absent in injured portions of the axon and, via mechanisms not entirely understood, SARM1 becomes activated and converts NAD⁺ into Nam; NAD⁺ is thus depleted and NMN accumulates in the injured axon via the action of NAMPT. Protective strategies used here interfere with the injury scenario via: genetically deleting SARM1 and thus preserving existing supplies of NAD⁺ (third graph); or blocking NAMPT with FK866, thus causing accumulation of uncatalyzed Nam and feedback inhibition of SARM1, which in turn conserves NAD⁺ (fourth graph). B, Sagittal images of cleared brainstems from SARM1^{+/+} (left panels) and SARM1^{-/-} (right panels), showing axonal abnormalities in three key portions of CST (CST×RST [top], decussation [middle], and cervical spinal cord [bottom]). There is a smaller number of axonal lesions in the SARM1^{-/-} brain. Scale bar, 60 μm. C, Counts of total numbers and regional densities of axonal lesions 24 h after injury. Error bars indicate mean±SEM. Group values were analyzed with Student's t test: **p<0.01; ***p<0.001.

FIG. 13: Protective effect of pharmacological interference with NMNAT2-SARM1 pathway at 24 h after injury. A, B, Sagittal images of cleared brainstems treated with vehicle (A) and FK866 (B), focusing on the CST and showing the concentration of axonal lesions in its medullary and upper cervical CST portions. There is a smaller number of axonal lesions in the FK866-treated brains. The CST×RST junction (1), decussation (2), and cervical spinal cord (3) of the vehicle and FK866-treated brains are further enlarged on the right. Scale bars: A, B, 300 μm; A1-A3, B1-B3, 60 μm. C, Left, Counts of total number of axonal lesions in CST 24 h after injury. Right, Densities of lesions in three key segments (CST×RST, pyramidal decussation [pyx], and cervical cord [cerv]). Error bars indicate mean±SEM. Group values were analyzed with Student's t test: **p<0.01; ***p<0.001.

FIG. 14: Course of secondary traumatic axonopathy in CST and effects of SARM1 deletion in preventing axon loss. (A-B) Gallyas silver (A) and Toluidine Blue-stained semithin material (B) show active degeneration at day 3, which subsides by day 7. (C) Super-resolution Airyscan microscopy (left) shows loss of CST axons in YFP-H mice at day 7 that remains stable when re-examined at day 21 (n=3 per group). Analysis of CST axon numbers on semithin sections (center) shows a significant difference in axonal survival between SARM1 KO (black bars; n=9) and WT mice (white bars; n=9) at day 3 (p<0.001) day 7 (p<0.000005). At later time points (Day 21, right), in which semithin counts are not reliable because of a compensatory increase in axon caliber and overestimation of axon numbers, unbiased EM-based axon counts (n=5 per group) show a sustained survival-promoting effect of SARM1 deletion (black bar) versus WT (white bars; p<0.05) (the Lorentzian curve center of the axon diameter frequency distribution with CI95% is: 0.333 to 0.376 μm for Sham versus 0.493 to 0.510 μm for Day 21 post injury).

FIG. 15: SARM1 blockade prevents CST-related motor deficits in injured mice. (A) To explore if the protective effect of SARM1 blockade on axons translates into preventing functional deficits, we established a CST-dependent skilled motor task, the pellet-reaching task. Here is a mouse performing to criterion to seize the pellet with its paw (left) versus swiping past the target (right). (B) Injured Sarm1 KO mice preserve fine motor control on pellet reaching, whereas injured WT mice deteriorate significantly (top). Likewise, FK866-treated injured mice maintain their pellet-reaching skill (bottom). Further experiments proposed here will extend observations to longer survival times. (** p<0.01, *** p<0.001).

FIG. 16: A drawing of a poly-dimethylsiloxane (PDMS)-based microfluidic device used for the experiments described in Example XII.

FIG. 17: NAMPT inhibitors FK866, STF118804, CHS-828, GPP78, were tested at the indicated doses 2 hours following axotomy and dose-response curves determined.

FIG. 18: A head to head comparison of different NAMPT inhibitors was conducted wherein NAMPT inhibitors were tested 2 hours following axotomy with the optimal doses.

FIG. 19: NAMPT inhibition was shown to afford similar protection to axons as SARM1 deletion up to 24 hours.

FIG. 20: Axotomized axons were treated with the NAMPT inhibitor FK866 at the time of injury or with delayed schedules as indicated (at 0,2,4,6 hours following injury) demonstrating an optimal therapeutic window of treatment onset within two to six hours of injury.

FIG. 21: Axotomized axons were treated with FK866 at the time of injury and NMN was added at different time points following axotomy (0, 2, 4 hours post axotomy). Reversal of the effect of NAMPT inhibition with demonstrated through addition of NMN.

FIG. 22: Axotomized axons were treated with FK866 with or without NAR. NAR augments the protective effect of NAMPT inhibition.

FIG. 23: DRGs were treated with the MAPK inhibitors (Sunitinib, Tozasertib or GNE3511) 8 hours before injury. Treatment effect was compared to control (DMSO) and delayed NAMPT inhibition.

FIG. 24: Protein changes for MKK4, JNK, SCG10, and DLK following axotomy in wild type, naïve axon fractions as a function of time.

### DETAILED DESCRIPTION

SARM1 is known to have a functional role in the regulation of neuronal survival/death. For example, murine SARM1 is reportedly predominantly expressed in neurons and is involved in the regulation of neuronal death in response to oxygen/glucose deprivation and exposure of neurons to the Parkinsonian neurotoxin 1-methyl-4-phenyl-1,2,3,6-tetrahydropyridine (MPTP) (see, Kim et al., JEM, 204: 2063-2074, 2007). A role for SARM has also been described in neuronal development (see, Yuan et al., J. Immunol., 184:6874-6881, 2010). SARM1 has been recently touted as the key executioner signal in the programmed degeneration of axons, the long efferent processes of neurons, known as Wallerian degeneration, that is triggered in both experimental axonal lesions and different classes of human disease (Conforti et al., Nat Rev Neurosci. 15(6):394-409, 2014; Gilley & Coleman, PLoS Biol., 26; 8(1):e1000300, 2010).

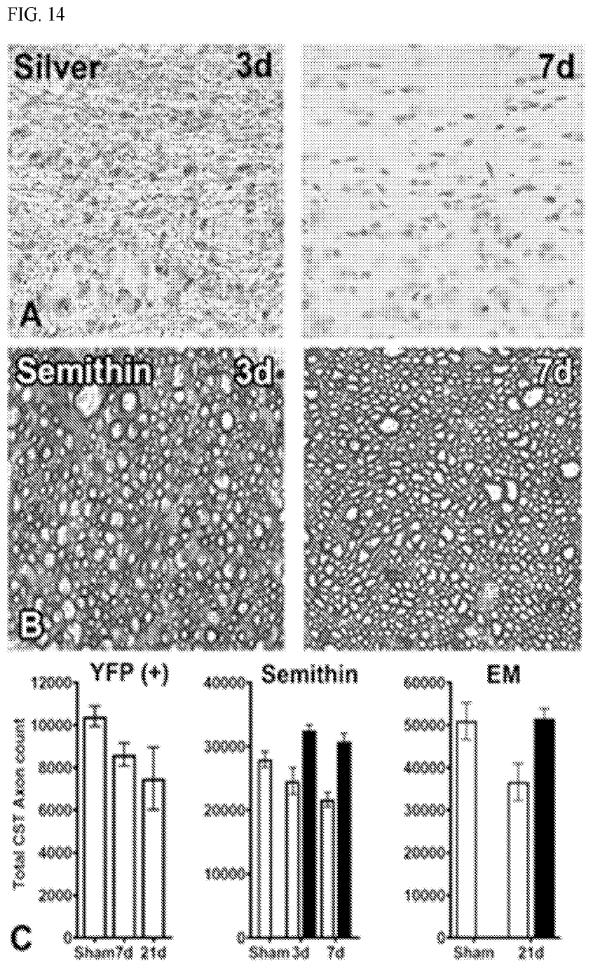

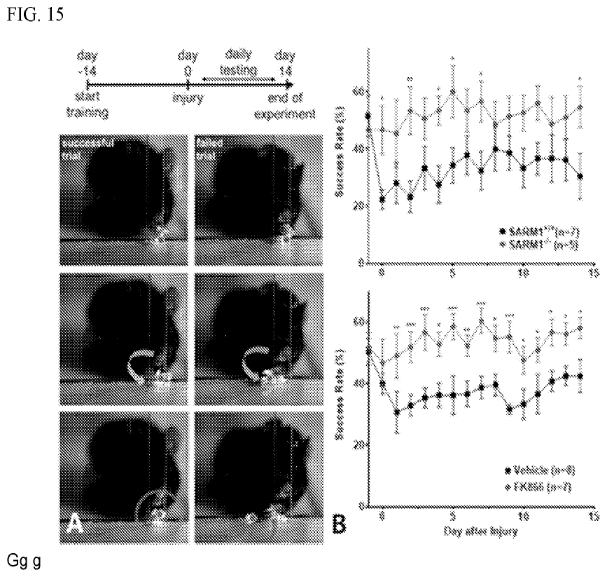

Experiments conducted during the course of developing embodiments for the present invention investigated the role of SARM1 in axonal degradation following TBI. In such experiments, Thy1-eYFP-H transgenic mice were subjected to IA to generate multifocal TAI accompanied by blood-brain barrier (BBB) disruption and neuroinflammatory responses by resident microglia or blood-borne macrophages. Experiments next focused on axonal pathology in the corticospinal tract (CST), a prototypical long-axon pathway, which were characterized with CLARITY and 2-photon microscopy at single-axon resolution. Based on CLARITY preparations, it was shown that the 3D distribution, precise magnitude, and the identity of TAI as progressive axonopathy. Experiments also explored the role of a recently proposed molecular pathway of axonal self-destruction in the early stages of traumatic axonopathy. This pathway is triggered when the axonal maintenance factor nicotinamide nucleotide adenylyltransferase 2 (NMNAT2) fails to reach the distal axon after injury, thus somehow initiating a sterile alpha and TIR motif containing 1 (SARM1)-dependent axonal degeneration program in which NAD⁺ metabolism plays a central role (see, Gilley J, et al., (2010) PLoS Biol 8:e1000300; Osterloh J M, et al., (2012) Science 337:481-484; Gerdts, et al., (2015) Science 348:453-457; Gerdts, et al., (2016) Neuron 89:449-460; Hill, et al., (2016) Trends Neurosci 39:311-324). To explore the significance of the NMNAT2-SARM1 pathway in traumatic axonopathy, experiments were conducted that interfered with SARM1 signaling by knocking out SARM1 or by inhibiting Nicotinamide phosphoribosyltransferase (NAMPT), an enzyme that is part of the NMNAT2-SARM1 signaling cascade, with a small-molecule inhibitor (FK866) (see, Essuman, et al., Neuron 93:1334-1343) thereby increasing levels of the SARM1 reaction product nicotinamide (Nam) that can serve as a feedback inhibitor of SARM1 and reduce levels of the NAMPT reaction product nicotinamide mononucleotide (NMN), metabolite with apparent toxic properties under conditions of injury. Such findings indicate that high-resolution anatomical strategies advance research on TAI by precisely localizing the injury, determining nature and severity, and allowing work into the molecular mechanisms of TAI-associated axonopathy as well as suggest clinically relevant molecular targets for therapy. As described herein and illustrated in FIGS. 14 and 15, these strategies not only lead to long-term preservation of injured axons but can also rescue functional and behavioral impairments.

In the absence of available SARM1 inhibitors, experiments described herein established the inhibition of the NMN synthesizing enzyme NAMPT, as an alternative therapeutic strategy in in vitro axotomy models. Wild type or SARM1 KO embryonic mouse dorsal root ganglion (DRG) cells were cultured in poly-dimethylsiloxane microfluidic devises for axon compartmentalization. Axons were cut with a razor blade and treated with the NAMPT inhibitor FK866 or vehicle at different time points. Images of axons were taken at different intervals and the degeneration index was calculated. Maximum protection against WD was observed with treatment at 2 hours post-injury which was similar to the protection afforded by SARM1 deletion. Importantly treatment after the onset of visible fragmentation was still effective in delaying further fragmentation. A group of other available NAMPT inhibitors (CHS-828, GPP78, STF118804, STF31) was also tested at different concentrations and showed comparably strong efficacy. Axonal preparations at different time points following axotomy were also used for metabolomic profiling and western blot assessment of injury related signals. Such experiments indicated that NAMPT inhibition by a variety of small molecules—some of which have already been tested in clinical trials—is an attractive molecular target and also therapeutic strategy against WD. Most importantly such experiments demonstrated that the molecular decision for WD is stochastic and temporally separated from the time of injury, effectively allowing for a clinically-relevant therapeutic window of intervention.

The above findings demonstrate that high-resolution neuroanatomical strategies optimized to serve as neuropathological tools reveal important features of TAI with biological implications, especially the progressive axonopathic nature of TAI and the role of the NMNAT2-SARM1 signaling and NAMPT inhibition in the early stages of axonopathy.

Such experiments further demonstrated that treatment of TAI through inhibition of SARM1 activity and/or expression and/or inhibition of NAMPT activity and/or expression is optimal with a therapeutic window of treatment onset within approximately two to six hours of injury (e.g., 3-5 hours, 2-5 hours, 2-7 hours, 2.5 hours to 6.5 hours, 1.5 to 6.5 hours, 1 to 7 hours, etc.), and that treatment should last at least approximately three days after injury (e.g., 2.5 days, 2.8 days, 3.1 days, 3.5 days, 4 days, 5 days, 7 days, 10 days, 14 days, 21 days, 100 days, etc).

Accordingly, the present inventors have succeeded in discovering that axonal degeneration can be diminished or prevented by interfering with NMNAT2/SARM1- and NAMPT-related signaling of axonal destruction in diseased and/or injured neurons through blocking SARM1 activity and/or expression and/or inhibiting NAMPT activity and/or expression in such diseased and/or injured neurons. Thus, one approach to preventing axonal degeneration can be by interfering with NMNAT2/SARM1 and/or NAMPT-related signaling of axonal destruction in injured mammalian axons through inhibiting SARM1 activity and/or expression and/or NAMPT activity and/or expression in such injured mammalian axons. The inhibition of SARM1 can be through direct action on SARM1 or by increasing the supply of the reaction product, Nam (e.g., through administration of FK866) which may act as a feedback inhibitor of SARM1. NAMPT inhibition is likely effective through other/additional mechanisms, such as the reduction of NMN. The inhibition of NAMPT can be through direct action on NAMPT (e.g., through administration of NAMPT inhibitors (e.g., FK866, CHS-828, GPP78, STF118804, STF31) (e.g., a chemically related or unrelated molecule that inhibits NAMPT). The inhibition of SARM1 and/or NAMPT results in a decrease in severity of axonal degeneration or prevention of axonal degeneration. Such treatment is optimized when administered within two to six hours of injury (e.g., 3-5 hours, 2-5 hours, 2-7 hours, 2.5 hours to 6.5 hours, 1.5 to 6.5 hours, 1 to 7 hours, etc.), and for a duration between three to fourteen days in vivo (e.g., in the case of a chronic neurodegenerative disease, such a treatment can be delivered in repeat epochs lasting at least 3 days each) (e.g., 2.5 days, 2.8 days, 3.1 days, 3.5 days, 4 days, 5 days, 7 days, 10 days, 14 days, 21 days, 100 days, etc).

Accordingly, the present invention involves methods and compositions for treating neuropathies and/or diseases or conditions characterized with axonal degradation.

The methods can comprise administering to a mammal an effective amount of a substance that interferes with NMNAT2-SARM1 signaling of axonal degradation through decreasing SARM1 activity in diseased and/or injured neurons.

The methods can comprise administering to a mammal an effective amount of a substance that interferes with NAMPT-NAM signaling of axonal degradation through decreasing NAMPT activity in diseased and/or injured neurons.

The methods can comprise administering to a mammal an effective amount of a substance that interferes with the NMNAT2-SARM1 and NAMPT signaling of axonal degradation pathway through decreasing SARM1 and NAMPT activity in diseased and/or injured neurons.

The methods can comprise administering to a mammal an effective amount of a substance that decreases SARM1 activity in diseased and/or injured neurons. Similarly, the methods can comprise administering to a mammal an effective amount of a substance that decreases SARM1 activity in diseased and/or injured neurons through increasing Nam levels in such diseased and/or injured neurons. As noted, experiments conducted during the course of the invention demonstrated that decreasing SARM1 activity and/or expression in diseased and/or injured neurons produces a decrease in axonal degeneration of injured neuronal cells compared to axonal degeneration that occurs in injured neuronal cells not treated with an agent for decreasing SARM1 activity and/or expression. Such decrease in axonal degeneration can also include a complete or partial amelioration of the injury to the neuronal cell body and the neuron as a whole.

The methods can comprise administering to a mammal an effective amount of a substance that decreases NAMPT activity in diseased and/or injured neurons. As noted, experiments conducted during the course of the invention demonstrated that decreasing NAMPT activity and/or expression in diseased and/or injured neurons produces a decrease in axonal degeneration of injured neuronal cells compared to axonal degeneration that occurs in injured neuronal cells not treated with an agent for decreasing NAMPT activity and/or expression. Such decrease in axonal degeneration can also include a complete or partial amelioration of the injury to the neuronal cell body and the neuron as a whole.

The methods can comprise administering to a mammal an effective amount of a substance that decreases SARM1 and NAMPT activity in diseased and/or injured neurons.

The present invention provides compositions and methods for modulating (e.g., inhibiting) SARM1 expression (e.g., protein and/or nucleic acid (mRNA) expression) and/or activity (e.g., protein activity). Such compositions and methods generally include targeting (e.g., specifically targeting) SARM1 DNA, mRNA, and/or protein to thereby modulate (e.g., inhibit) SARM1 mRNA and/or protein expression and/or function. In some instances, targeting (e.g., specifically targeting) SARM1 can include targeting (e.g., specifically targeting) SARM1 in a neuron (including in the neuronal cell body), in an axon, in a synapse, and/or in a dendrite. In some embodiments, SARM1 can be targeted through increasing Nam levels in a relevant cell as Nam is a feedback inhibitor of SARM1.

The present invention provides compositions and methods for modulating (e.g., inhibiting) NAMPT expression (e.g., protein and/or nucleic acid (mRNA) expression) and/or activity (e.g., protein activity). Such compositions and methods generally include targeting (e.g., specifically targeting) NAMPT DNA, mRNA, and/or protein to thereby modulate (e.g., inhibit) NAMPT mRNA and/or protein expression and/or function. In some instances, targeting (e.g., specifically targeting) NAMPT can include targeting (e.g., specifically targeting) NAMPT in a neuron (including in the neuronal cell body), in an axon, in a synapse, and/or in a dendrite.

Compositions for modulating SARM1 and/or NAMPT expression and/or activity can include, but are not limited to, one or more of: small molecules, inhibitory nucleic acids, antibodies, and inhibitory peptides. For example, one or more of a small molecule, an inhibitory nucleic acid, an anti-SARM1 and/or anti-NAMPT antibody, and/or an inhibitory nucleic acid can be used to target (e.g., specifically target) SARM1 and/or NAMPT in a neuron (including in the neuronal cell body), in an axon, in a synapse, and/or in a dendrite, thereby modulating (e.g., decreasing) (e.g., inhibiting) SARM1 and/or NAMPT to reduce axonal and/or synaptic degradation in the subject.

Suitable small molecules include small molecules that inhibit SARM1 expression and/or activity directly, indirectly, or both directly and indirectly. Suitable small molecules include small molecules that bind (e.g., bind specifically) to SARM1 and thereby inhibit SARM1 expression and/or activity, and/or small molecules that do not bind to SARM1 or that bind to SARM1 with low affinity, but that inhibit SARM1 expression and/or activity by binding to a component of the SARM1 signaling pathway upstream or downstream of SARM1.

In some embodiments, composition and pharmaceutical compositions comprising FK866 are provided as FK866 is known to increase NAM levels in cells, which acts as a feedback inhibitor of SARM1 activity. Thus, FK866 acts to decrease SARM1 activity through increasing NAM levels.

Suitable small molecules include small molecules that inhibit NAMPT expression and/or activity directly, indirectly, or both directly and indirectly. Suitable small molecules include small molecules that bind (e.g., bind specifically) to NAMPT and thereby inhibit NAMPT expression and/or activity, and/or small molecules that do not bind to NAMPT or that bind to NAMPT with low affinity, but that inhibit NAMPT expression and/or activity by binding to a component of the NAMPT signaling pathway upstream or downstream of NAMPT.

In some embodiments, composition and pharmaceutical compositions comprising an agent capable of inhibiting NAMPT activity and/or expression are provided. In some embodiments, the agent is a NAMPT inhibitor. In some embodiments, the NAMPT inhibitor is selected from FK866, CHS-828, GPP78, STF118804, and STF31. In some embodiments, the agent is any type of small molecule compound or pharmaceutical formulation that is capable of inhibiting NAMPT activity and/or expression. In various embodiments, the agent decreases NAMPT activity through inhibiting NAMPT genetic expression (e.g., gene therapy) (e.g., genetic ablation of NAMPT expression).

Inhibitory nucleic acids suitable for use in the methods described herein include inhibitory nucleic acids that bind (e.g., bind specifically) to SARM1. Also encompassed are inhibitory nucleic acids that bind (e.g., bind specifically) to a component of the SARM1 signaling pathway upstream or downstream of SARM1. Exemplary inhibitory nucleic acids include, but are not limited to, siRNA and antisense nucleic acids. Exemplary treatment options involving administration of inhibitory nucleic acids include for purposes of inhibiting SARM1 activity and/or expression include, but are not limited to, RNAi treatment, siRNA treatment, antisense treatment, and targeted gene expression inhibition.

Inhibitory nucleic acids suitable for use in the methods described herein include inhibitory nucleic acids that bind (e.g., bind specifically) to NAMPT. Also encompassed are inhibitory nucleic acids that bind (e.g., bind specifically) to a component of the NAMPT signaling pathway upstream or downstream of NAMPT. Exemplary inhibitory nucleic acids include, but are not limited to, siRNA and antisense nucleic acids. Exemplary treatment options involving administration of inhibitory nucleic acids include for purposes of inhibiting NAMPT activity and/or expression include, but are not limited to, RNAi treatment, siRNA treatment, antisense treatment, and targeted gene expression inhibition.

The present disclosure also includes methods that include the use or administration of antibodies and antibody fragments that bind (e.g., bind specifically) to SARM1 and thereby inhibit SARM1 activity in a neuron. Antibodies and antibody fragments that bind (e.g., bind specifically) epitopes expressed (e.g., specifically expressed) on the surface of a neuron such that when the epitope is bound by the antibody SARM1 expression and/or activity is reduced are also included in the present disclosure.

The present disclosure also includes methods that include the use or administration of antibodies and antibody fragments that bind (e.g., bind specifically) to NAMPT and thereby inhibit NAMPT activity in a neuron. Antibodies and antibody fragments that bind (e.g., bind specifically) epitopes expressed (e.g., specifically expressed) on the surface of a neuron such that when the epitope is bound by the antibody NAMPT expression and/or activity is reduced are also included in the present disclosure.

Also included in the present disclosure are methods that include the use or administration of inhibitory peptides that bind (e.g., bind specifically) to SARM1 or interact with SARM1 and thereby inhibit SARM1 activity and/or expression in a neuron. Such peptides can bind or interact with an epitope on SARM1 and/or with a SARM1 domain.

Also included in the present disclosure are methods that include the use or administration of inhibitory peptides that bind (e.g., bind specifically) to NAMPT or interact with NAMPT and thereby inhibit NAMPT activity and/or expression in a neuron. Such peptides can bind or interact with an epitope on NAMPT and/or with a NAMPT domain.

The present invention provides methods for treating a subject with or at risk of any condition that is associated with axonal degradation (e.g., a neuropathy; a neurodegenerative disease; a complication of diabetes) with a composition disclosed herein to target and thereby modulate (e.g., inhibit) SARM1 and/or NAMPT to reduce axonal degradation in the subject.

The present invention contemplates the treatment of neuropathies through inhibiting SARM1 and/or NAMPT activity and/or expression. Neuropathies can include any disease or condition involving neurons and/or supporting cells, such as for example, glia, muscle cells, fibroblasts, etc., and, in particular, those diseases or conditions involving axonal damage. Axonal damage can be caused by traumatic injury or by non-mechanical injury due to diseases or conditions and the result of such damage can be degeneration or dysfunction of the axon and loss of functional neuronal activity. Disease and conditions producing or associated with such axonal damage are among a large number of neuropathic diseases and conditions. Such neuropathies can include peripheral neuropathies, central neuropathies, and combinations thereof. Furthermore, peripheral neuropathic manifestations can be produced by diseases focused primarily in the central nervous systems and central nervous system manifestations can be produced by essentially peripheral or systemic diseases.

Peripheral neuropathies involve damage to the peripheral nerves and such can be caused by diseases of the nerves or as the result of systemic illnesses. Some such diseases can include diabetes, uremia, infectious diseases such as AIDs or leprosy, nutritional deficiencies, vascular or collagen disorders such as atherosclerosis, and autoimmune diseases such as systemic lupus erythematosus, scleroderma, sarcoidosis, rheumatoid arthritis, and polyarteritis nodosa. Peripheral nerve degeneration can also result from traumatic, i.e. mechanical damage to nerves as well as chemical or thermal damage to nerves. Such conditions that injure peripheral nerves include compression or entrapment injuries such as glaucoma, carpal tunnel syndrome, direct trauma, penetrating injuries, contusions, fracture or dislocated bones; pressure involving superficial nerves (ulna, radial, or peroneal) which can result from prolonged use of crutches or staying in one position for too long, or from a tumor; intraneural hemorrhage; ischemia; exposure to cold or radiation or certain medicines or toxic substances such as herbacides or pesticides. In particular, the nerve damage can result from chemical injury due to a cytotoxic anticancer agent such as, for example, a vinca alkaloid such as vincristine. Typical symptoms of such peripheral neuropathies include weakness, numbness, paresthesia (abnormal sensations such as burning, tickling, pricking or tingling) and pain in the arms, hands, legs and/or feet. The neuropathy can also be associated with mitochondrial dysfunction. Such neuropathies can exhibit decreased energy levels in axons.

The peripheral neuropathy can also be a metabolic and endocrine neuropathy which includes a wide spectrum of peripheral nerve disorders associated with systemic diseases of metabolic origin. These diseases include diabetes mellitus, hypoglycemia, uremia, hypothyroidism, hepatic failure, polycythemia, amyloidosis, acromegaly, porphyria, disorders of lipid/glycolipid metabolism, nutritional/vitamin deficiencies, and mitochondrial disorders, among others. The common hallmark of these diseases is involvement of peripheral nerves by alteration of the structure or function of myelin and axons due to metabolic pathway dysregulation.

Neuropathies also include optic neuropathies such as glaucoma; retinal ganglion degeneration such as those associated with retinitis pigmentosa and outer retinal neuropathies; optic nerve neuritis and/or degeneration including that associated with multiple sclerosis; traumatic injury to the optic nerve which can include, for example, injury during tumor removal; hereditary optic neuropathies such as Kjer's disease and Leber's hereditary optic neuropathy; ischemic optic neuropathies, such as those secondary to giant cell arteritis; metabolic optic neuropathies such as neurodegenerative diseases including Leber's neuropathy mentioned earlier, nutritional deficiencies such as deficiencies in vitamins B12 or folic acid, and toxicities such as due to ethambutol or cyanide; neuropathies caused by adverse drug reactions and neuropathies caused by vitamin deficiency. Ischemic optic neuropathies also include non-arteritic anterior ischemic optic neuropathy.

The present invention contemplates treatment of neurodegenerative diseases through inhibition of SARM1 and/or NAMPT activity and/or expression. Neurodegenerative diseases that are associated with neuropathy or axonopathy in the central nervous system include a variety of diseases. Such diseases include those involving progressive dementia such as, for example, Alzheimer's disease, senile dementia, Pick's disease, and Huntington's disease; central nervous system diseases affecting extrapyramidal movements such as, for example, Parkinson's disease, motor neuron diseases and progressive ataxias such as amyotrophic lateral sclerosis; demyelinating diseases such as, for example multiple sclerosis; viral encephalitides such as, for example, those caused by enteroviruses, arboviruses, and herpes simplex virus; and prion diseases. Mechanical injuries such as glaucoma or traumatic injuries to the head and spine can also cause nerve injury and degeneration in the brain and spinal cord. In addition, ischemia and stroke as well as conditions such as nutritional deficiency and chemical toxicity can cause central nervous system “neuropathies”.

The present invention provides methods for treating subjects with or at risk of diabetic neuropathy with the compositions disclosed herein to target and thereby modulate (e.g., inhibit) SARM1 to reduce axonal degradation in the subject.

The term “treatment” as used herein is intended to include intervention either before or after the occurrence of neuronal injury. As such, a treatment can prevent neuronal injury by administration before a primary insult to the neurons occurs as well as ameliorate neuronal injury by administration after a primary insult to the neurons occurs. Such primary insult to the neurons can include or result from any disease or condition associated with a neuropathy. “Treatment” also includes prevention of progression of neuronal injury. “Treatment” as used herein can include the administration of drugs and/or synthetic substances, the administration of biological substances such as proteins, nucleic acids, viral vectors and the like as well as the administration of substances such as neutraceuticals, food additives or functional foods.

The methods and compositions of the present invention are useful in treating mammals. Such mammals include humans as well as non-human mammals. Non-human mammals include, for example, companion animals such as dogs and cats, agricultural animals such live stock including cows, horses and the like, and exotic animals, such as zoo animals.

Treatment can include administration of an effective amount of one or more of an agent to target and thereby modulate (e.g., inhibit) SARM1 and/or NAMPT in a neuron, in an axon, in a synapse, and/or in a dendrite. Compositions can be administered by any means that results in inhibition of SARM1 and/or NAMPT in a neuron (including in the neuronal cell body), in an axon, in a synapse, and/or in a dendrite. For example, compositions can be administered systemically and/or locally. Systemic administration can include use of compositions that target neurons in the CNS and/or PNS. Local administration can include administration of compositions to a defined region of the CNS and/or PNS, including, but not limited to, an injury site.

Administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, oral, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

It is also contemplated that certain formulations containing the compositions that decrease SARM1 and/or NAMPT activity and/or expression are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methylcellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated such as to provide rapid, sustained, or delayed release of the active ingredients after administration to the patient by employing procedures well known in the art. The formulations can also contain substances that diminish proteolytic degradation and promote absorption such as, for example, surface-active agents.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention further provides methods for selecting or identifying compositions, compounds, or agents that modulate (e.g., inhibit) SARM1 and/or NAMPT expression and/or activity, for use in the treatment of any condition or disorder associated with axonal degradation (e.g., Wallerian degeneration). Exemplary compositions can include compositions that interact (e.g., specifically interact) with SARM1 and/or NAMPT DNA, mRNA, and/or protein to thereby modulate (e.g., inhibit) SARM1 and/or NAMPT mRNA and/or protein expression and/or function. Methods include, for example, screening for candidate compounds using one or more of: in silico, in vitro and/or in culture (e.g., using high-throughput screening methods); and/or animal models (e.g., to test/verify candidate compounds as compounds that inhibit SARM1 and/or NAMPT). Compounds can also be evaluated in clinical trial, e.g., for use in human subjects. Techniques for performing such screening methods are known in the art and/or are described herein.

Compounds screened can include, but are not limited to, small molecules, inhibitory nucleic acids, antibodies, and inhibitory peptides. For example, commercial libraries of compounds (e.g., small molecules) can be screened using in vitro high-throughput screening methods. Such libraries include libraries containing compounds (e.g., small molecules) previously approved for use in human subjects (e.g., approved by the Federal Drug Administration).

The present invention also provides kits comprising a topical composition comprising a SARM1 and/or NAMPT inhibiting agent and instructions for administering the compound to an animal. The kits may optionally contain other therapeutic agents.

### EXPERIMENTAL

The following examples are provided to demonstrate and further illustrate certain preferred embodiments of the present invention and are not to be construed as limiting the scope thereof.

### Example I

This example describes the diffuse, multifocal nature of IA injury.

Animal models of TBI are featured by various degrees of axonal injury, but only in few cases there is a clear distinction of primary axonal injury from secondary Wallerian degeneration (see, Xiong Y, et al., (2013) Nat Rev Neurosci 14:128-142). Part of the problem is that most popular models of TBI either are solely based on focal lesions or have substantial focal components that directly cause neuronal cell death and thus are not primary axonopathies. In rodents, one of the best-characterized models of diffuse TBI with primary axonal injury is impact acceleration (IA) (see, Marmarou A, et al., (1994) J. Neurosurg 80:291-300). Using a modification of this model for the mouse cranium, primary traumatic axonopathy in multiple CNS pathways, including the visual system, corticospinal tract (CST), corpus callosum, medial and lateral lemniscus, and the cerebellar white matter has been demonstrated (see, Xu, et al., (2016) Exp Neurol 275:436-449).

Another challenge in modeling TAI is that available methods to demonstrate axonal injury are primarily based on axonal transport blockade markers, such as APP, that label damaged axons temporarily (see, Stone J R, et al., (2000) Brain Res 871:288-302) or silver degeneration stains that may label only degenerating axons (see, Blackstad T W, et al., (1981) Experimental neuroanatomy. In: Neuroanatomical tract-tracing methods (Reimer L, Robards M J, eds), pp 1-53. New York, N.Y.: Springer). In addition, the near-absence of the third dimension in routine histological sections allows only a limited sampling of tissue and leads to underestimation of the extent of axonal abnormalities. The advent of transgenic mice that express the yellow fluorescent protein (YFP) under Thy1 promoter offers great promise for a sensitive and specific labeling of axonal pathology (see, Greer, et al., (2011) J Neurosci 31:5089-5105; Greer, et al., (2013) Acta Neuropathol 126:59-74; Hånell, et al., (2015) Acta Neuropathol 129:317-332). More importantly, the invention of methods that render the brain tissue transparent, such as CLARITY (see, Chung, et al., (2013) Nature 497:332-337; Tomer, et al., (2014) Nat Protoc 9:1682-1697), makes it possible, for the first time, to microscopically study whole axon tracts and visualize, analyze, and quantitate the full 3D extent of axonal pathology.

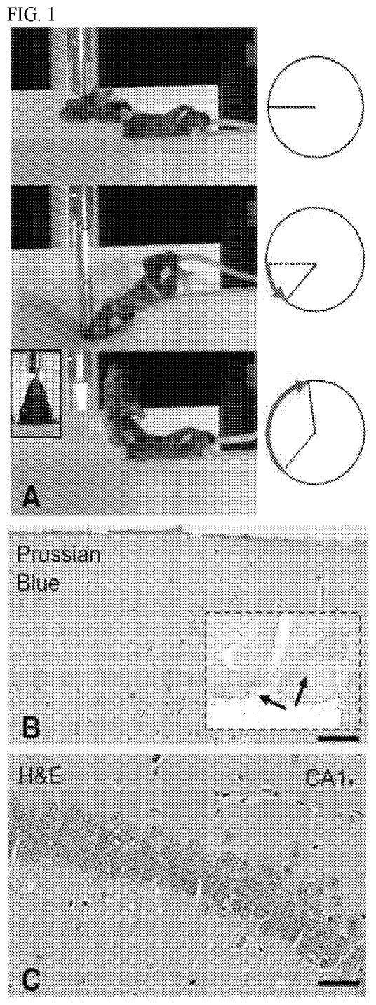

To characterize the IA model from a biomechanical perspective, the injury process was imagined with a high-frame rate camera, shooting at 20.000 frames per second for the whole duration of the movement of the mouse head (FIG. 1A). Head motion was confined to the sagittal plane and was consistent among IA sessions. As shown in FIG. 1A, the head was subjected to both translational and angular (rotational) acceleration, first downward into the foam bed of the IA apparatus and then upward to a nearly vertical position. With 60 g×1 m impact, the duration of head movement was ˜150 ms and the resulting velocity was 4.1 m/s. The placement of the metallic disc on the skull prevented fractures in all cases.

To assess the possible effects of IA on brain parenchyma at the impact site (area under the disc) tissues were processed with H&E for general pathology, and Perls' Prussian blue for ferric iron (parenchymal blood). Overall, no evidence of cytoplasmic eosinophilia or nuclear pyknosis of cortical neurons was found. Cortical structure was intact everywhere, including motor, sensory, and parietal cortical areas under the disc. No cortical staining with Perls' iron was found, but a weak diffuse signal in the mesencephalic flexure of brainstem was seen (FIG. 1B). In addition, the CA1 field of hippocampus was free of eosinophilic neurons, a pattern consistent with absence of significant brain hypoxia (FIG. 1C).

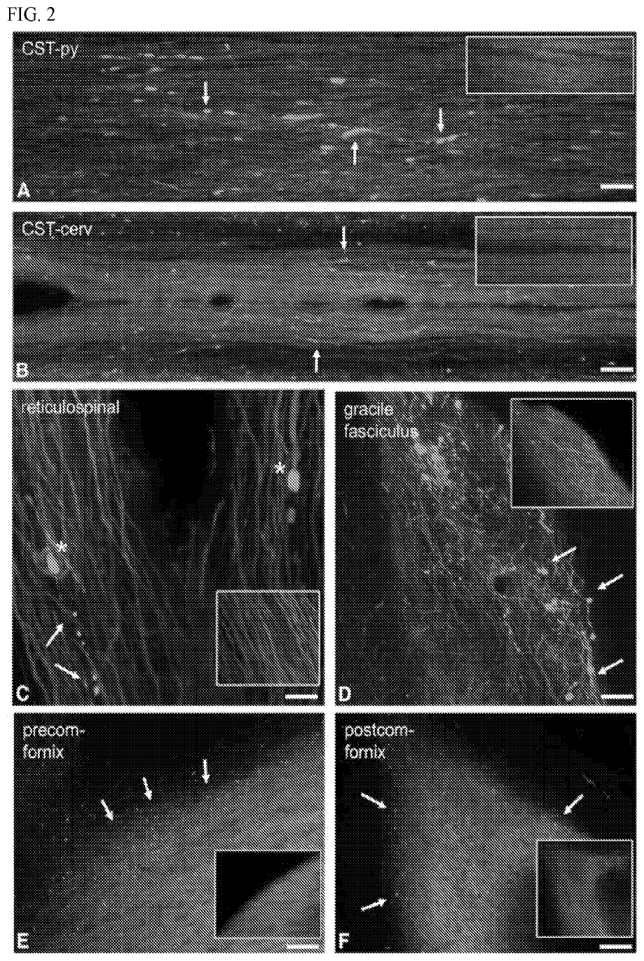

Using brain sections from YFP-H mice and fluorescence microscopy, axonal abnormalities at multiple sites in the forebrain, diencephalon, midbrain, medulla, and the cervical spinal cord were found (FIG. 2). These abnormalities consisted mainly of varicosities or axon bulbs and, more rarely, axonal vacuolation or trails of intensely fluorescent small axon fragments suggestive of Wallerian degeneration. In an approximately rostrocaudal order, major white matter tracts exhibiting such abnormalities included the precommisural and postcommisural fornix, the habenulo-interpeduncular tract, the lower CST, the reticulospinal tract (RST), and the gracile fasciculus. In view of the fact that in the YFP-H transgenic line used here only certain populations of projection neurons are labeled, it is likely that more white matter tracts become involved in IA injury within the parameters used in the experiments (see, Xu L, et al., (2016) Exp Neurol 275: 436-449).

Various axonal tracts involved in IA injury show somewhat distinct pathologies. Axonal profiles in the CST, measuring 3-6 μm in diameter, are described in the next section. For example, in the RST, fragmentation profiles suggestive of Wallerian degeneration are evident as early as 24 h after injury (FIG. 2C) and increase at 48 h. Many RST axons show large varicosities (8-10 μm in diameter) and some show vacuolations. The gracile fasciculus develops numerous characteristic large varicosities (12-15 μm in diameter) both in the course of this tract in the dorsal spinal cord-medulla and at the terminal fields in the gracile nucleus; the number and size of these lesions cause them to stand out in sagittal sections of the brainstem (FIG. 2D). In the fornix, axonal abnormalities take the form of numerous small (1-μm-diameter) spheroids distributed near or at the terminal fields of the precommissural and postcommissural fornix in the septum and mammillary bodies, respectively (FIGS. 2E and F). Lesions are present as early as 3 h after injury, and they increase in number at 24 and 48 h.

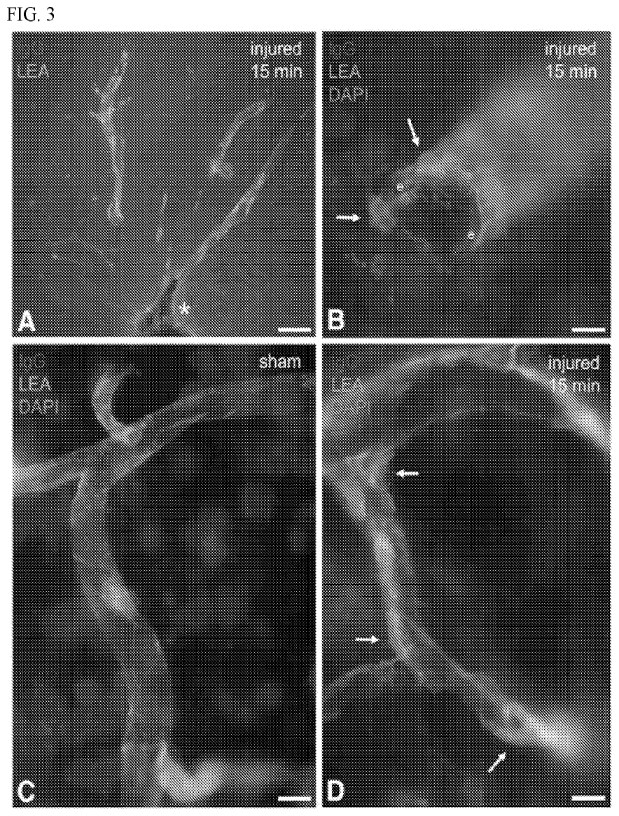

To further characterize the IA injury, the presence and early time course of microvascular injury (i.e., BBB breakdown) was established. To this goal, endothelial cells with LEA were labeled via intracardial injection immediately before perfusion fixation and then immunostained sections with mouse anti-IgG antibody at early time points after injury (i.e., 15 min, 1 h, and 2 h). In well-perfused subjects, IgG immunoreactivity in brain parenchyma indicates leakage of plasma from brain capillaries and thus serves as a marker of BBB degradation (see, Wang J T, et al. (2015) Proc Natl Acad Sci USA 112:10093-10100; Xu L, et al., (2016) Exp Neurol 275:436-449). IgG immunoreactivity outside brain capillaries was found as early as 15 min after IA injury (FIG. 3). IgG leakage was restricted to the brainstem and was especially severe in the region of mesencephalic flexure, a pattern suggesting a primary effect of IA on that part of the brain (FIG. 3A). Under high magnification, sections with IgG and LEA labeling show unambiguous IgG localization outside the endothelium (FIG. 3B-D).

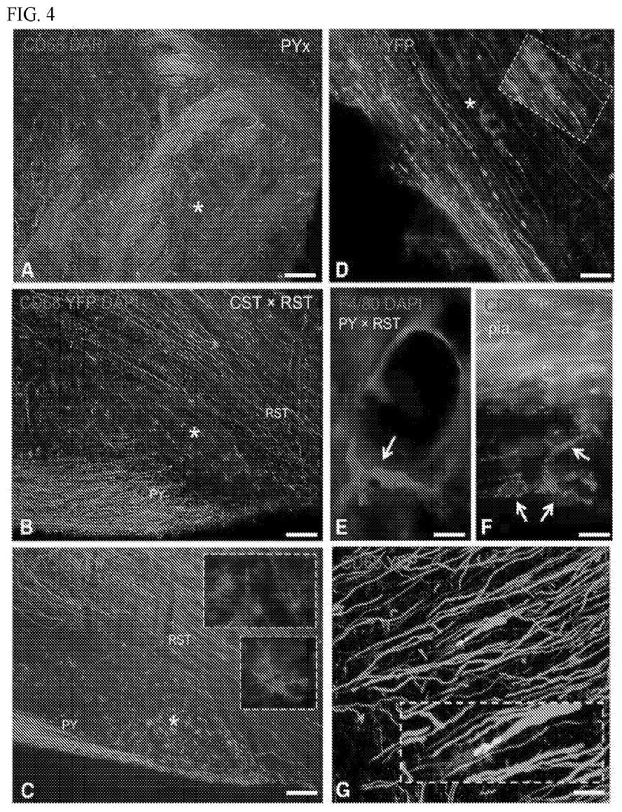

A key aspect of traumatic axonopathy is the neuroinflammatory macrophagic response. To characterize the main features of this response in our model, IHC was performed for the representative microglial and macrophage protein epitopes P2Y12, CD68, and F4/80 48 h after IA injury (FIG. 4). P2Y12, a metabotropic ADP receptor, is constitutively expressed in abundance in microglial cells (see, Haynes S E, et al. (2006) Nat Neurosci 9:1512-1519; Kobayashi K, et al. (2008) J Neurosci 28:2892-2902). In the material, P2Y12 immunoreactivity was present in resting microglial cells throughout the brain. P2Y12 (+) cells with hypertrophic cell bodies characteristic of activated microglial cells were numerous in certain brainstem sites, including the area of inferior olive at the crossing of pyramids with the RST and the pyramidal decussation (i.e., regions with dense axonal lesions) (FIG. 4C). These activated cells had thick, short processes instead of the ramified appearance of resting microglia and formed clusters of two to four cells (FIG. 4C, inset 1) or had stout thorny processes typical or phagocytes (FIG. 4C, inset 2) (see, Yamada J and Jinno S (2013) J. Comp Neurol 521:1184-1201). In addition to the CST, activated microglia was present in fornix, corpus callosum, gracile nucleus, and cerebellum, all areas associated with axonal injury after IA (see, Xu L, et al. (2016) Exp Neurol 275:436-449). In the CST, activated microglia and peripheral macrophages abound at the pyramidal decussation and CST×RST, where there is a majority of axonal abnormalities, but were not detected in CST axon segments that do not show pathology, such as cortex, internal capsule, peduncles, or pons. Activated microglial cells were also labeled with IHC for CD68, a lysosomal protein that is highly expressed in activated microglia. As in the case of P2Y12, CD68 labeling was especially strong in lower pyramids and the pyramidal decussation (FIGS. 4A, B and G). Experiments also explored the presence of infiltrating blood-borne macrophages with the marker F4/80. Such F4/80 (+) macrophages were absent in the brain parenchyma of uninjured mice. Forty-eight hours after IA injury, F4/80-positive macrophages had made their appearance in the brain parenchyma, forming clusters around capillaries close to injured CST axons (FIGS. 4D and E).

In conclusion, IA produces diffuse primary axonal injury accompanied by BBB disruption and neuroinflammation comprised of both resident and blood-borne macrophage response. The initial shearing stress is especially evident in the brainstem, as shown by the anatomical distribution of early BBB impairments. The Thy1-eYFP-H transgenic line offers remarkable detail on the location of axonal injury, the distinct cytologies of axonal lesions in various tracts, as well as the longitudinal evolution of axonopathy.

### Example II

This example describes the nature and severity of axonal abnormalities in the CST.

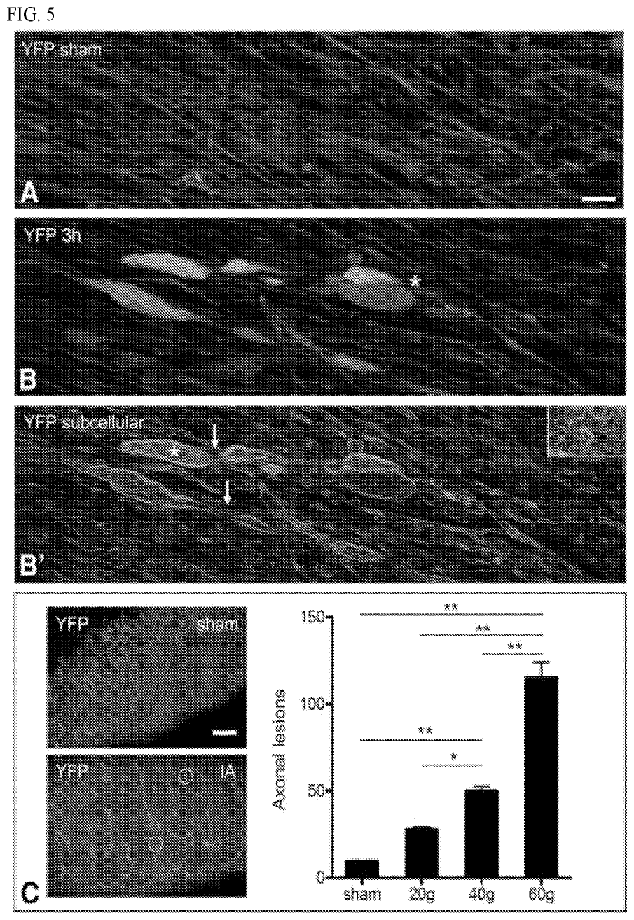

Based on observations in YFP-H mice, axonal abnormalities in the CST occurred almost exclusively at brainstem and spinal levels. Such abnormalities in cortex, internal capsule, or cerebral peduncles were not seen. Most lesions were axonal varicosities or end bulbs at the level of the pyramids and the pyramidal decussation (FIG. 2A). In many cases, multiple varicosities or varicosities and bulbs coexisted within the same axon. Abnormalities in the spinal cord were seen along the dorsal CST (FIG. 2B). Many axons formed classical retraction balls (i.e., disconnected spherical formations that are distinct from bulbs that may be still attached to distal atrophic axons). Confocal microscopy and edge detection analysis confirmed the presence of multiple varicosities on single axons and showed high concentration of spherical organelles at sites of axonal bulging (FIG. 5B-B′). Based on counts of YFP (+) axonal varicosities in cross sections of pyramids, the severity of axonal pathology in the CST is related to IA burden (FIG. 5C). There is a 2× and 3× increase in the number of lesions when weight in the IA device advances from 20 to 40 and from 40 to 60 g, respectively.

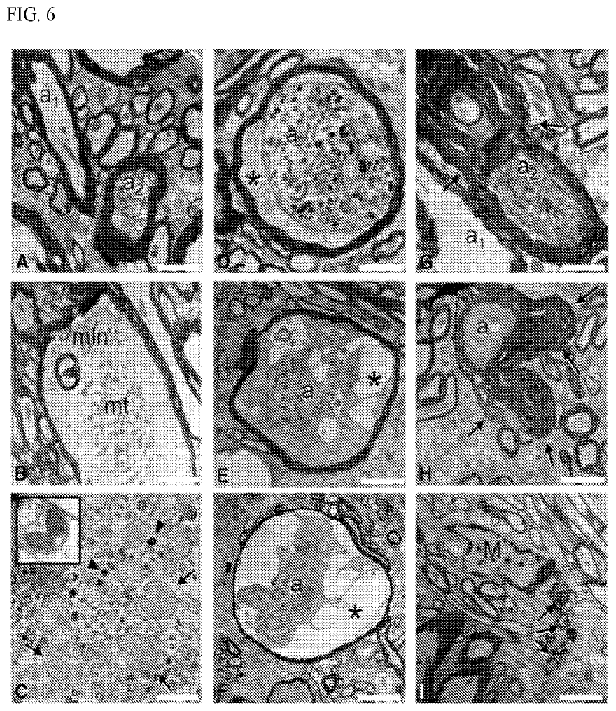

The ultrastructural analysis of tissue at the level of lower pyramids/pyramidal decussation shows that axonal swellings identified with fluorescent microscopy correspond to sites of axonal degeneration. Degenerative changes include severe alterations in overall axon structure, organelles, the axoplasm, and the myelin sheath (FIG. 6). In such cases, axons are condensed with degraded or absent neurofilament or microtubule architecture. Other axons are filled with tightly packed electron-dense bodies and damaged, swollen mitochondria without clearly delineated cristae (FIG. 6C). Many axons are abnormally large, with vacuoles in the axoplasm and separation of axoplasm from the axolemma. Myelin pathology was also present, with myelin intrusions, excess myelin figures, and abnormal thickening of the myelin sheath. Macrophages with dark inclusions were frequently apposed to degenerating axons (FIG. 61). Also, intact axons were interspersed among degenerating axons, a pattern characteristic of DAI.

In summary, the CST is severely afflicted during IA of the head, and severity of axonopathy can be titrated based on severity of impact.

### Example III

This example demonstrates retrograde changes in corticospinal neurons: c-Jun phosphorylation and atrophy.

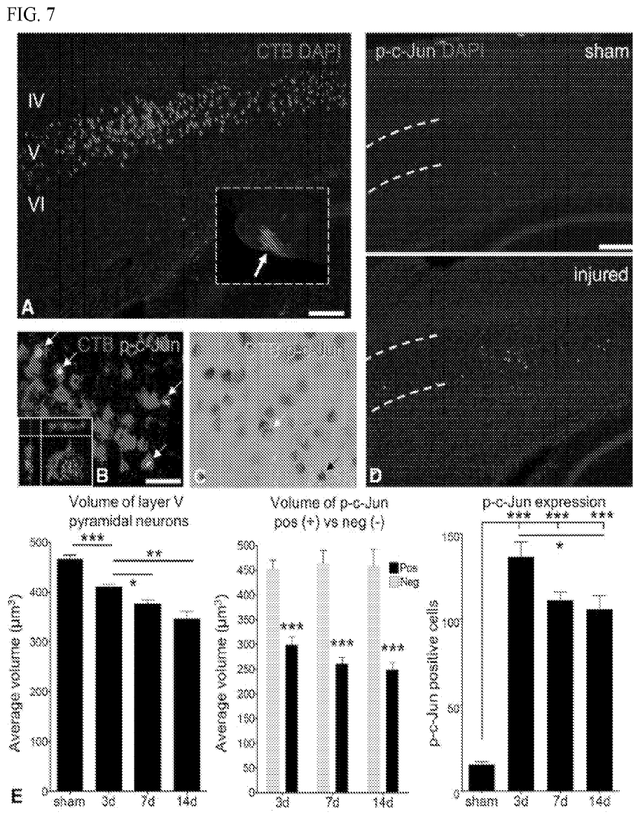

To identify pyramidal neurons whose axons were injured in the lower pyramidal tract, the presence and cytology of corticospinal neurons in frontal neocortex that also expressed phosphorylated c-Jun was explored (FIG. 7). The cell bodies of neurons projecting in the CST were identified by retrograde filling with the tracer CTB injected into the ponto-medullary junction (FIG. 7A). Phosphorylation of c-Jun in the nuclei of corticospinal neurons was examined with immunohistochemistry (FIGS. 7B and C). Corticospinal neurons were studied at 3, 7, and 14 d after injury, covering the period from the induction of c-Jun phosphorylation early on to later changes in perikaryal volume. Three specific trends were explored: time course of volume changes in CTB-labeled perikaryal, time course of phosphorylated c-Jun expression, and rates of p-c-Jun expression in atrophic and normal layer V pyramidal neurons.

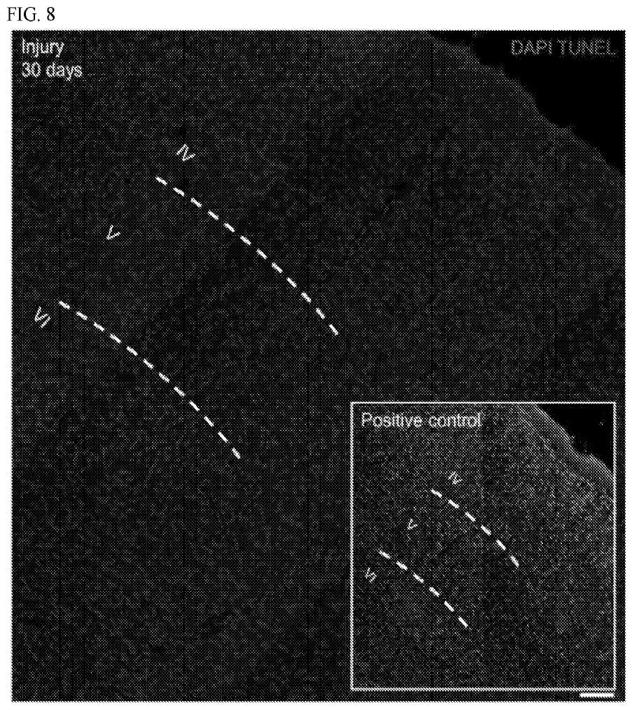

It was found that 10 d after injection into the ponto-medullary portion of CST, CTB strongly labels layer V pyramidal neurons in frontal neocortex (FIG. 7A). Retrogradely labeled pyramidal profiles in subjects injured with IA were significant smaller than pyramidal profiles in sham animals at all three time points; for example, at 3 d, average volume was reduced by 12%, whereas at 14 d average volume was reduced by 26% (FIG. 7E). Phosphorylated c-Jun immunoreactivity was upregulated after axonal injury, and it was most profound at 3 d after injury with the signal decreasing slightly at later time points (FIGS. 7D and E). In addition, at all three time points, perikaryal volume was significantly smaller in neurons expressing p-c-Jun compared with the ones with normal volume, a pattern suggesting a relationship between p-c-Jun expression and perikaryal atrophy (FIG. 7C-E). Based on the negative TUNEL staining for neuronal cell death at 3, 7, 14, and 30 d after IA, layer V pyramidal neurons do not die up to a month after injury (FIG. 8).

In conclusion, CST neurons with injured axons become atrophic after injury, but they do not undergo cell death.

### Example IV

This example describes CLARITY-based quantitative analysis of axonal injury in the CST.

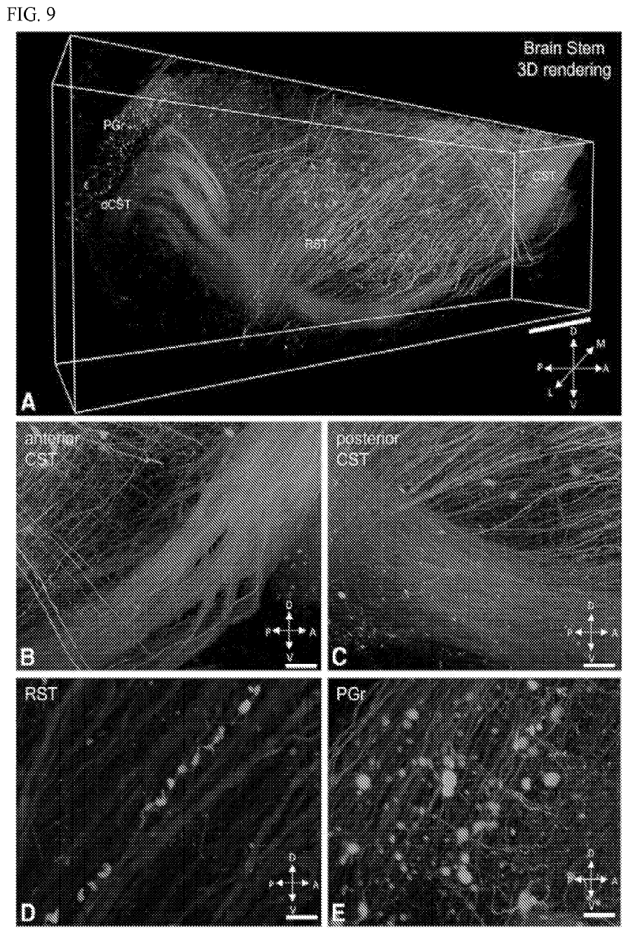

To characterize the full 3D extent of axonal injury in the CST, the brainstem was rendered transparent with CLARITY and examined cleared tissues with high-resolution 2-photon microscopy coupled with a CLARITY-optimized objective. These strategies allowed resolving of individual axons at 25× magnification and generated a sharp 3D visualization of axonal tracts coursing in the brainstem from the level of the lower cerebral peduncle-pons to the dorsal columns of the cervical spinal cord (FIG. 9). Based on the YFP expression pattern in Thy1-YFP-H mice, the main pathways that can be visualized are the CST, the RST, and the gracile fasciculus. All three tracts developed axonal abnormalities and these lesions were sharply visualized across the entire thickness of the brainstem, allowing for an impressive reconstruction of the location and magnitude of the injury effect (FIG. 9A). Moreover, the combination of 2-photon microscopy, HyD detectors in the nondescanned position, and a long-working-distance objective with high numerical aperture revealed important pathological features of individual lesions (FIG. 9B-E).

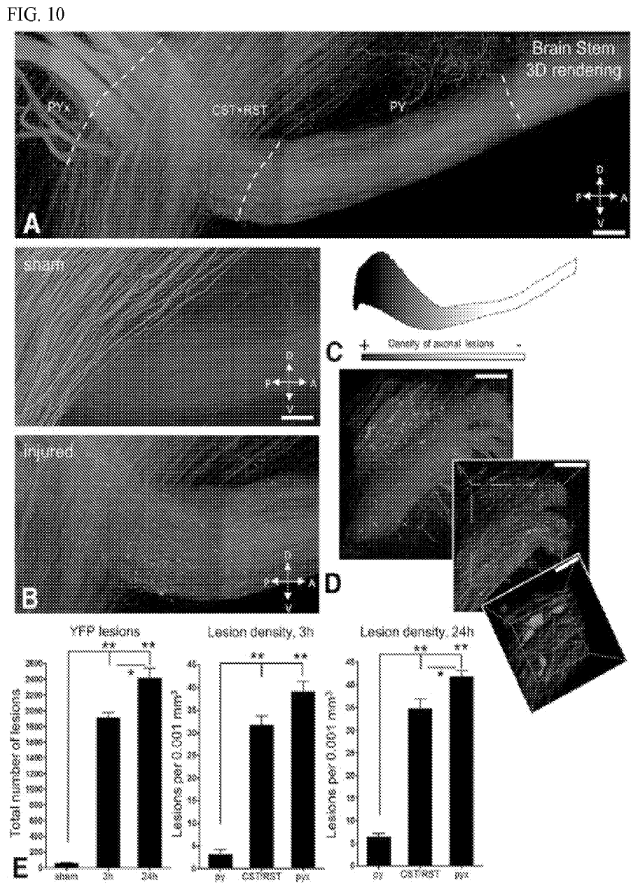

The high-resolution 3D reconstruction of brainstem over a distance of 6 mm provided raw data for counting the total number of axonal abnormalities in distinct CST segments in the medullary pyramids, at the crossing of CST with the RST (CST×RST), and at the level of pyramidal decussation (FIG. 10A-D). Using such data, it was found that the total number of axonal abnormalities increased significantly from 3 to 24 h after injury (FIG. 10E). The density of axonal abnormalities varies among regions: it is low at the main body of the pyramid, high at CST×RST, and even higher at the level of the pyramidal decussation; density increases across regions from 3 to 24 h after injury, but the differential involvement of these regions remains the same (FIG. 10E). The rostrocaudal progression of lesion severity is impressive (FIG. 10C).

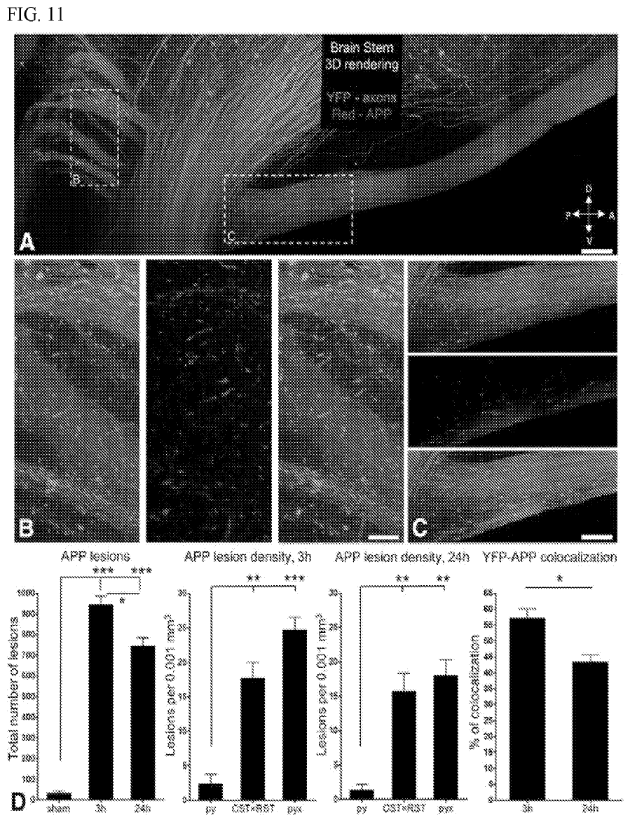

CLARITY-based IHC for APP, the standard molecular marker of axonal injury, was used to compare between the number of lesions detected with this marker and YFP (+) lesions in the brainstem at two time points after injury: i.e., 3 and 24 h (FIG. 11A-D). APP (+) lesions were numerous at 3 h and decreased significantly at 24 h (FIG. 11D), in contrast to YFP (+) lesions that increased from 3 to 24 h (FIG. 10E). The total number of APP (+) lesions was smaller than YFP (+) lesions across all time points (FIG. 10E, left vs. FIG. 11D, left). The density of APP (+) abnormalities was counted in the pyramids, at CST×RST, and at the pyramidal decussation. Areas with highest density were the decussation, followed by CST×RST, a pattern similar to the one encountered with YFP axonal abnormalities. These data show that YFP reveals a larger number of axonal abnormalities than APP labeling and has a different time course, perhaps due to the accumulation of structural pathology in the first 24 h period.

Such results indicate the presence of progressive axonopathy that is maximal at the pyramidal decussation and they establish CLARITY as a robust quantitative methodology for studying TAI in the mouse brain. Markers of axonal injury that detect anterogradely transported proteins (i.e., APP) may be less representative of the problem than YFP, which is encoded by a stably expressed transgene with robust labeling of the axoplasm.

### Example V

This example demonstrates that genetic interference with SARM1 or pharmacological interference with NAMPT signaling ameliorates early axonal pathology.

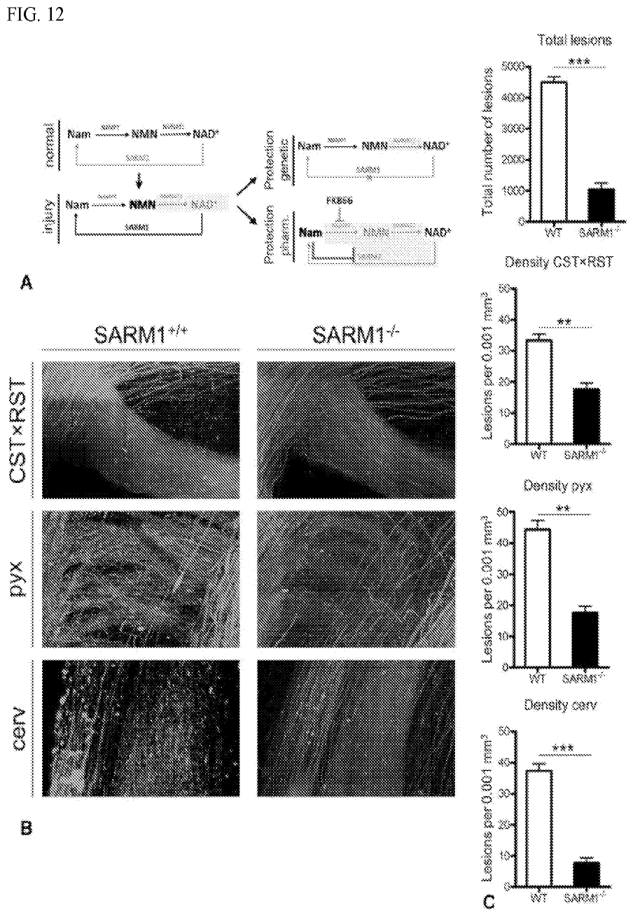

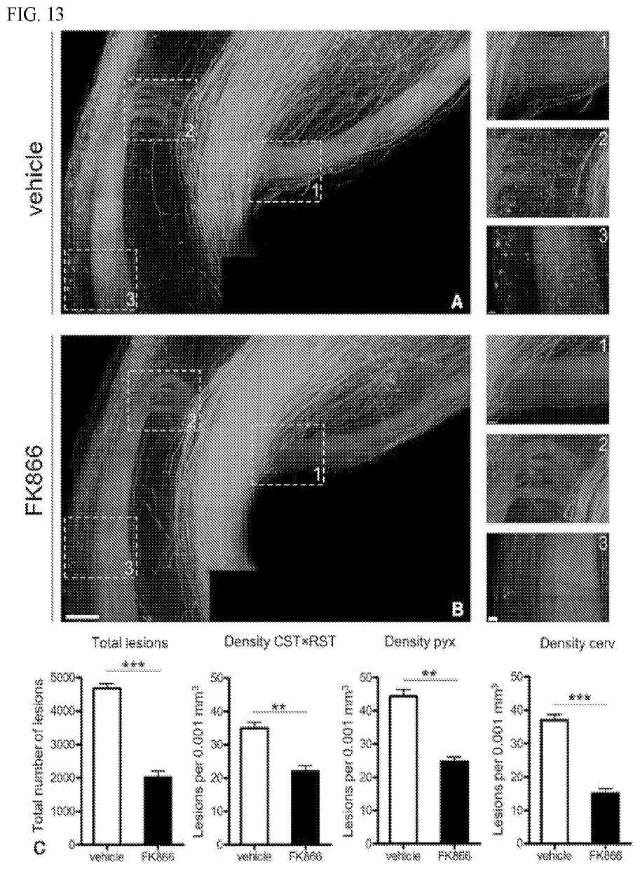

Experiments were conducted that took advantage of the resolution and quantitative capabilities of CLARITY to explore the role of the NMNAT2-SARM1 pathway of axonal self-destruction in traumatic axonopathy (FIG. 12, FIG. 13). The anterograde transport blockage in injured axons prevents the labile axon maintenance factor NMNAT2 from reaching the distal axon, thus triggering SARM1-depended axonal degeneration (see, Gilley J, and Coleman M P (2010) PLoS Biol 8: e1000300; Osterloh J M, et al., (2012) Science 337:481-484; Gerdts J, et al., Neuron 89:449-460; Hill C S, et al., (2016) Trends Neurosci 39:311-324; Summers D W, et al., (2016) Proc Natl Acad Sci USA 113:E6271-E6280; Walker U, et al., (2017) eLife 6:e22540; Essuman K, et al., (2017) Neuron 93: 1334-1343). SARM1 is a NADase that is dormant in uninjured axons in the presence of NMNAT2. When NMNAT2 is depleted after injury, SARM1 becomes activated and consumes axonal NAD⁺, thus presumably causing axonal degeneration. The products of the enzymatic activity of SARM1 are Nam, ADP-ribose and cyclic ADP-ribose. Of these products, Nam is thought to inhibit and regulate SARM1 activity via negative feedback (FIG. 12A).

To establish the involvement of the NMNAT2-SARM1 related signaling in the early stages of axonal degeneration after TAI, we generated Thy1-eYFP-H mice in which SARM1 was deleted (Thy1-eYFP-H/SARM1^{-/-}). These mice have precisely the same pattern of YFP expression as Thy1-eYFP-H mice. Thy1-eYFP-H/SARM1^{-/-} and Thy1-eYFP-H/SARM1^{+/+} were injured with IA and brains were processed with CLARITY at 24 h after injury (FIG. 12B). The total number of axonal lesions in the CST from pons to cervical cord and regional densities of abnormalities as in the previous section was counted. It was found that SARM1^{-/-} mice had a significantly lower total number of CST lesions compared with SARM1^{+/+} mice, and significantly lower concentrations of abnormalities at key CST segments (CST×RST, pyramidal decussation, and cervical cord). Density of lesions was greater at the cervical cord and decussation than CST×RST (FIG. 12C).

To explore the role of NMNAT2-SARM1 related signaling early in the course of traumatic axonopathy with pharmacological means, experiments used FK866 to decrease levels of NMN and raise levels of Nam in the axonal compartment (see, Essuman K, et al., (2017) Neuron 93: 1334-1343). Nam participates in NAD⁺ biosynthesis through an intermediate, NMN, a conversion catalyzed by NAMPT. In an injured axon, SARM1 activation depletes NAD⁺ rapidly and the Nam produced is being converted to NMN very efficiently (see, Sasaki Y, et al., (2009) J. Neurosci 29:6526-6534). Blocking NAMPT (e.g., with FK866) decreases levels of NMN that may have toxic properties in injured axons and also increases Nam, change that may interfere with SARM1 activity (FIG. 12A). In the experiment, IA-injured Thy1-eYFP-H mice were treated with FK866 or vehicle and brains were processed with CLARITY (FIG. 13). The total number of axonal abnormalities in the CST from the pons to cervical spinal cord was counted 24 h after injury as in the previous section of the paper, and focusing on lower segments of the CST, including CST×RST, pyramidal decussation, and cervical cord (FIGS. 13A and 13B). It was discovered that FK866-treated mice had a significantly lower number of axonal lesions compared with vehicle-treated mice both in total and per anatomical segment, with a trend toward bigger difference in the cervical cord (FIG. 13C).

In summary, the activation of the NMNAT2-SARM1 related signaling is a prominent mechanism of traumatic axonopathy with a significant role in early axonal degeneration. The robust qualitative and quantitative advantages of CLARITY make it a powerful tool in elucidating molecular mechanisms of axonal degeneration.

### Example VI

This example describes the materials and methods for Examples I-V. Experimental animals.

The experimental subjects were 5-week-old male C57BL/6 mice (strain code: 027, Charles River Laboratories, RRID), 5-week-old male Thy1-eYFP-H transgenic mice (catalog #003782, The Jackson Laboratory), and 5-week-old male Thy1-eYFP-H/SARM1^{-/-} transgenic mice. The transgenic line Thy1-eYFP-H expresses Thy1-driven YFP in specific populations of CNS and PNS neurons, including pyramidal neurons in layer V of motor and sensory neocortex, with superb delineation of the CST throughout its entire course from cortex to spinal cord (see, Porrero C, et al., (2010) Brain Res 1345:59-72). The expression of YFP was ascertained by genotyping for the Tg (Thy1-YFP) HJrs allele. All animals received humane care in compliance with the Guide for the care and use of laboratory animals (see, National Academy Press (2011) Guide for the care and use of laboratory animals, Ed 8. Washington, D.C.: National Academy) and based on procedures approved by the Animal Care and Use Committee of the Johns Hopkins Medical Institutions. Animals were housed in a vivarium with 12 h light/12 h dark cycles and given access to pellet food and water ad libitum.

### IA Model of TBI

IA injury was produced under gas anesthesia with 2% isoflurane. Under aseptic conditions and with the subject in a small-animal stereotactic frame, a 5-mm-diameter steel disc was glued on the exposed cranium between bregma and lambda to prevent skull fracture. The mouse was then placed prone on a foam bed under a hollow Plexiglas tube and secured with tape as described previously (see, Xu L, (2016) Exp Neurol 275:436-449). Injury was produced by dropping a brass weight from a height of 1 m through the Plexiglas tube onto the disk. In all cases except the impact dose-response study outlined below, that weight was 60 g. The foam bed was pulled immediately at the rebound of the weight off the metal disc, such as to prevent a second impact. The metal disc was then removed, scalp incision was closed with surgical staples, and the animal returned to cage for full recovery. Subjects with skull fractures confirmed with a surgical microscope were excluded from the study. Sham-operated animals were subject to the same procedures without the weight drop component. For a video recording of the IA injury, a high-frame rate camera, Fastcam SA5 (Photron) was used, shooting at 20,000 frames per second for the entire duration of the injury.

General Neuropathology and Study of Axonal Injury with Fluorescence or Confocal Microscopy.

At the appropriate time points, C57BL/6 or Thy1-eYFP-H mice were transcardially perfused with freshly depolymerized PFA (4% in 0.1 m PBS, pH 7.4). Brains were postfixed in the same fixative for various periods of time. For general neuropathological characterization of the injury, we used brain tissues from C57BL/6 animals perfused 7 d after injury that had been postfixed for 3 d in PFA. Tissues were embedded in paraffin and coronal sections (10 μm) were stained for cresyl violet, Prussian blue, and H&E.

For characterization of the axonal injury, brain tissues from Thy1-eYFP-H mice perfused 3, 24, and 48 h after injury was used. These tissues were postfixed overnight (4° C.) in PFA, then saturated with 20% glycerol containing 5% DMSO, frozen in dry ice, and sectioned at the sagittal or coronal plane (40 μm). Sagittal brain sections from injured and control Thy1-eYFP-H mice (n=10 per group) were used to characterize injured YFP-labeled tracts based on the presence of axonal swellings. Sections were studied with epifluorescence or confocal microscopy. For confocal microscopy, sections between the midsagittal plane and a plane 0.5 mm lateral to midline that contain the entire CST were imaged with a LSM-700 unit at 40×. In some cases, imaging was done with care not to saturate the fluorescent signal at the axonal swelling sites and the ImageJ edge detection function was applied after acquisition to visualize cytosolic vesicles and organelles.

### Blood-Brain Barrier (BBB) Study.

Brain endothelial cells from injured and control C57BL/6 mice (n=5 per group) were labeled by transcardial injection of 150 μl of Lycopersicon Escelentum lectin (LEA) conjugated with DyLight 488 (Vector Laboratories catalog #DL-1174) 3 min before perfusion with 4% PFA. Sagittal sections were blocked with 5% donkey serum in PBS with 0.2% Triton X-100 (PBST) for 3 h and then incubated in donkey anti-mouse IgG conjugated with Cy3 (Jackson ImmunoResearch Laboratories catalog #715165150) in blocking solution for 2 h. After washing, sections were counterstained with DAPI and imaged using an Axioplan microscope (Carl Zeiss) or a LSM-700 confocal microscope.

### Impact Dose-Response Study.

Thy1-eYFP-H mice injured with sham procedures or 20, 40, and 60 g IA (n=5 per group) were perfused with 4% PFA 24 h after injury, and brainstems were sectioned coronally on a sliding microtome. Four sections per subject through the pyramids were mounted on a microscopic slide and imaged with an LSM-700 confocal microscope. Axonal swellings were counted by blinded investigators using ImageJ.

### CLARITY-Based Brain Clearing Optimized for Axonal Injury Detection, Immunohistochemistry, Imaging, and Quantification.

For brain clearing, a modified CLARITY method was used (see, Chung K, et al., (2013) Structural and molecular interrogation of intact biological systems. Nature 497: 332-337). In summary, injured and control Thy1-eYFP-H mice were transcardially perfused with 4% PFA at 3 and 24 h after injury (n=3 per group). Brains were incubated in a hydrogel solution containing 4% PFA, 2% acrylamide (Bio-Rad catalog #1610140), 0.025% bis-acrylamide (Bio-Rad catalog #1610142), and 0.25% VA-044 initiator (Wako catalog #VA-044) in 0.1 m PBS for 3 din 4° C. Tissues were then degassed in a vacuum desiccator and polymerized at 37° C. for 3 h. After removal of the excess gel, brains were transferred in 50 ml of a clearing solution consisting of 4% SDS in 200 mm boric acid, pH 8.5, for 2 weeks at 37° C. until fully transparent. After clearing, brains were incubated in PBST for 2 d. CLARITY-based immunohistochemistry (IHC) for amyloid precursor protein (β-APP or APP) used a polyclonal antibody against the C terminus of the protein that is widely used as a marker of axonal injury (1:60; Thermo Fisher Scientific catalog #51-2700, RRID): after the clearing step, brains were incubated for 5 din APP/PBST (37° C.) followed by a 2 d PBST wash and then incubation in anti-rabbit IgG AlexaFluor-568 (1:60; Thermo Fisher Scientific catalog #A-11011, AB 143157) in PBST for 5 d at 37° C. After a final wash, brains were transferred to FocusClear (CelExplorer catalog #FC-101) and mounted for imaging.

To achieve single-axon resolution to detect individual axonal lesions, two-photon microscopy using HyD detectors in the nondescanned position and CLARITY-optimized, long-working-distance, high-numerical-aperture 25× objectives were optimized. This key modification of the original CLARITY protocol was achieved with a TCS SP8 MP multiphoton microscope coupled with HyD NDD detectors (Leica Microsystems) and a motorized 6 mm working-distance, CLARITY-optimized objective (Leica Microsystems). For each case, the whole mouse brain was cleared and the brainstem imaged for further analysis. The imaging of each mouse brainstem took 24 h and generated ˜120 GB of data per animal.

For 3D visualization and quantitative analysis of axonal injury of cleared brainstems, we used the software suite Imaris (Bitplane). The spot detection analysis was based on the size of axonal lesions in the 3D space. Because of the size of imaging data a 128 GB-RAM workstation was used. For the detection and quantification of axonal swellings based on YFP and APP, the spot detection function for the whole CST volume from the pons to the pyramidal decussation or cervical spinal cord was used.

### Genetic Deletion of SARM1.

Sarm1^{-/-} mice (see, Szretter K J, et al. (2009) J Virol 83:9329-9338) were obtained from and cross-bred in house with Thy1-eYFP-H mice; SARM1 deletion was confirmed by PCR. Thy1-eYFP-H/SARM1^{-/-} and Thy1-eYFP-H/SARM1^{+/+} mice were injured with 60 g×1 m IA (n=3 per group). Experimental sample sizes were predetermined based on power analysis from previous CLARITY experiments (see, FIG. 10). All experimental subjects have been included in the study. Counts were performed by a blinded investigator. Subjects were killed 24 h after injury and brain/spinal cord tissues were processed with CLARITY as in previous section.

Pharmacological Interference with the NMNAT2-SARM1 Pathway.

Thy1-eYFP-H mice were injured with 60 g×1 m IA (n=3 per group), and treated with FK866 (10 mg/kg i.p.) (Sigma-Aldrich catalog #F8557) or vehicle starting immediately after injury and then continuing every 6 h until 24 h after injury, when the experiment was terminated (see, Busso N, et al. (2008) PLoS One 3: e2267; Bruzzone S, et al. (2009) PLoS One 4: e7897; Nahimana A, et al. (2009) Blood 113:3276-3286; Van Gool F, et al. (2009) Nat Med 15:206-210). Experimental sample sizes were determined with power analysis from previous CLARITY experiments, as in the previous section. All experimental subjects were included in the study. Counts were performed by a blinded investigator. Mice were killed 24 h after injury and tissues were processed with CLARITY.

### Electron Microscopy.

Injured and control Thy1-eYFP-H mice (n=3 per group) were transcardially perfused with 4% PFA/2% glutaraldehyde 24 h after injury. Brains were postfixed overnight in the same solution (4° C.). Tissue cubes through the lower pyramids were further fixed in PFA/glutaraldehyde solution for 5 d (4° C.), treated with 2% osmium tetroxide in 0.1 m sodium cacodylate, pH 7.4, and then stained en bloc with 2% uranyl acetate. Blocks were then dehydrated, embedded in Embed 812 (Electron Microscopy Sciences), and sectioned on a Reichert Jung Ultracut E microtome. Semithin sections were stained with 1% toluidine blue. Thin sections were stained with uranyl acetate followed by lead citrate and photographed on a Carl Zeiss Libra 120 electron microscope equipped with a Veleta (Olympus) camera.

### Retrograde Tracing of Corticospinal Pyramidal Neurons to Identify Injured Cell Bodies.

Recombinant cholera toxin (subunit B) conjugated to AlexaFluor-594 (Thermo Fisher Scientific catalog #C22842) was injected stereotactically into the CST at the level of the ventral pons (4.1 mm caudal to bregma, 0.7 lateral to midline, 5.25 mm ventral to pial surface) of C57BL/6 mice (see, Paxinos G, and Franklin K B (2012) Paxinos and Franklin's the mouse brain in stereotaxic coordinates. Amsterdam, the Netherlands: Elsevier). Tracer (0.5 μl of a 1.0 mg/ml solution in PBS) was pressure injected with glass micropipettes as described previously (see, Capurso S A, et al. (1997) Deafferentation causes apoptosis in cortical sensory neurons in the adult rat. J Neurosci 17:7372-7384; Clatterbuck R E, et al., (1998) Peripheral nerve grafts exert trophic and tropic effects on anterior thalamic neurons. Neurobiol Dis 5:17-26). Two weeks later, mice were subjected to IA or sham injury and 3, 7, and 14 d after injury mice were perfused with 4% PFA (n=5 per injury or sham×survival time). To colocalize p-c-Jun and CTB immunoreactivities, we performed chromogen-based, dual-label IHC or fluorescent IHC for p-c-Jun only. In the former case, we used immunoperoxidase-DAB for CTB and alkaline-phosphatase for p-c-Jun. Briefly, sections were blocked in 5% normal donkey serum and 0.2% Triton X-100 (2 h, room temperature) and then incubated in primary antibodies against CTB (1:1000; List Laboratories catalog #703) and p-c-Jun (Ser 63) (1:100; Cell Signaling Technology catalog #9261) (overnight, 4° C.). For immunoperoxidase labeling, after incubation in biotinylated donkey anti-goat IgG antibody (1:200; Jackson ImmunoResearch Laboratories catalog #705065147) and then in avidin-biotin HRP (Vectastain Elite ABC Kit; Vector Laboratories catalog #PK-6100), sections were developed with a standard DAB reaction. For alkaline-phosphatase staining, sections were incubated in ImmPress-AP anti-rabbit IgG polymer detection kit (Vector Laboratories catalog #MP5401) and developed with a blue alkaline-phosphatase substrate (Vector Laboratories catalog #SK5300). In the case of p-c-Jun immunofluorescence, after blocking in 5% normal donkey serum and 0.2% Triton X-100 (2 h, room temperature), sections were incubated in the p-c-Jun antibody (1:100) overnight at 4° C. After washing and incubation in a secondary Cy2 conjugated donkey anti-rabbit IgG (1:200; Jackson ImmunoResearch Laboratories catalog #711225152) for 2 h in room temperature, sections were counterstained in DAPI and coverslipped in DPX. Fluorescent sections were imaged with an Axioplan microscope (Carl Zeiss) and a LSM-700 confocal microscope (Carl Zeiss).

Phosphorylated-c-Jun (+) layer V neurons were counted on 6 serial sections. Volumes of layer V pyramidal neurons were stereologically estimated with the nucleator probe by blinded investigators using StereoInvestigator software (MBF Bioscience).

### TUNEL.

Control and injured C57BL/6 mice were perfused at 3, 7, 14, and 30 d (n=3 per group) with 4% PFA. Brains were sectioned at the sagittal plane (40 μm). For TUNEL staining of dying neurons, we used TMR red (Sigma-Aldrich catalog #12156792910) as per the manufacturer's instructions. Sections incubated with DNase I (Sigma-Aldrich catalog #AMPD11KT) served as positive controls. After TUNEL reactions, sections were coverslipped in DPX and imaged with an LSM-700 confocal microscope (Carl Zeiss).

### Immunohistochemistry for Protein Markers of Immune Cells.

Control and injured Thy1-eYFP-H mice (n=5 per group) were perfused with 4% PFA 48 h after injury. Sagittal brain sections (40 μm) were processed in series for fluorescent IHC addressing select immune cell markers: P2Y12 (1:1000; AnaSpec catalog #AS55043A), CD68 (1:200; Abbiotec catalog #250594), and F4/80 (1:300; Abcam catalog #ab16911). Immunofluorescence was performed essentially as described in previous sections. Secondary antibodies were all used in a 1:200 concentration and included Cy3 donkey anti-rabbit IgG (Jackson ImmunoResearch Laboratories catalog #711165152) for P2Y12 and CD68 antibodies and Cy3 donkey anti-rat IgG (Jackson ImmunoResearch Laboratories catalog #712165153) for the F4/80 antibody. Sections were counterstained in DAPI and coverslipped in DPX. Sections were imaged with an Axioplan microscope (Carl Zeiss) and a LSM-700 confocal microscope (Carl Zeiss).

### Statistical Analyses.

Statistical analyses were performed using Prism 4 (GraphPad Software) and results are expressed as mean±SEM. Student's t test was used for single comparisons. One-way ANOVA with Tukey post hoc testing was used for multiple comparisons. In all cases, statistical significance was set at p<0.05.

### Example VII

Experiments were conducted to characterize the effects of SARM1 deletion on corticospinal axon degeneration after traumatic brain injury (impact-acceleration) at late stages of axonopathy (to explore the enduring effects of interventions). First, using Gallyas silver and Toluidine Blue-stained semithin material active degeneration of the CST was found at day 3, which subsided by day 7. Super-resolution Airyscan microscopy shows loss of CST axons in YFP-H mice at day 7 that remains stable when re-examined at day 21. These data using silver, semithin, and high-resolution confocal microscopy show rise and then cessation of traumatic axonal degeneration within the first week post-injury. The number of normal axons at the cervical spinal cord level on toluidine blue-stained semithin sections was counted at days 3 and 7 and, by EM, at day 21 in both wild type and SARM1 OK mice. On semithin sections, a significant difference in axonal survival between SARM1 KO (n=9) and WT mice (n=9) was found at day 7. At later time points (Day 21), thin sections were used as semithin counts are not reliable due to a compensatory increase in axon caliber and overestimation of numbers of axons. At this time point, unbiased EM-based axon counts (n=5 per group) show a sustained survival-promoting effect of Sarm1 deletion. These data show a long-standing protection of injured corticospinal axons by SARM1 blocking strategies in the case of TBI. On a separate experiment, axons were also examined and counted with a new type of high-resolution confocal microscopy (Airyscan) that allows single-axon resolution and very precise counts as well as estimates of thickness of axons.

### Example VIII

This example describes the methods for Example VII.

Study mice were anesthetized with isoflorane and 1% chloral hydrate. Then they were perfused with PBS, then 4% paraformaldehyde and 2% glutaraldehyde in 0.1M phosphate buffer. After excision, spinal cords were fixed overnight in fresh fix. One-millimeter thick blocks from cervical spinal cord (C2-C4) were taken. These samples were rinsed briefly then post-fixed in 1% osmium tetroxide overnight. Samples were dehydrated in ethanol, embedded in Embed 812 (Electron Microscopy Services, Hatfield, Pa.) then flat mounted in silicon molds. Then they were polymerized at 60° C. for 48 hours. One-micron (semithin) sections were cut transversely and stained with toluidine blue Imaging was done. Normal Axons were counted under 100× magnification using stereological methods taking into account both density and cross-sectional surface of the axonal bundle in question (CST). Thin sections were also prepared for EM microscopy. The defined CST area was sampled at 8000× in an unbiased and systematic manner. Stereological estimates of total numbers of axons from both semithin and thin preparations were used in statistical calculations for determining group size effect, using Student's t-test in GraphPad Prism 4.

Axonal counts were also conducted in Thy-eYFP-H mice with high-reolution confocal miscroscopy. Mice were intracardially perfused with 4% paraformaldehyde in 0.1M phosphate buffer. Spinal cords were dissected and fixed overnight in fresh fixative. Fifty-micrometer sections were cut at the transverse plane through the cervical spinal cord (C6). Sections were mounted and images were acquired using super-resolution Airyscan microscopy. Images were extracted, binarized and analysed with ImageJ to estimate the total number of YFP+ axons in the CST.

### Example IX

Experiments were conducted to characterize the effects of SARM1 deletion or pharmacological SARM1 blockade on corticospinal-dependent motor deficits after traumatic brain injury (impact-acceleration). This was tested on the pellet-reaching task, i.e. the golden-standard to assess skilled function of the forelimbs in rodents relevant to a similar task used to assess fine motor function in humans. Specifically, experiments trained mice for 2 weeks before TBI in order to reach peak performance in the task. At that point the trained mice were injured with impact-acceleration. Experiments next measured their performance every day for two weeks post injury and comparisons were made between groups of mice administered FK866 and vehicle treated mice (control group). When the drug was administered continuously for 14 days post injury a significant preservation of skilled motor function was observed compared to vehicle-treated mice.

### Example X

This example describes the methods for Example IX.

Mice were placed under food restriction or 2 days to lose approximately 10% of their body weight. Following this, the shaping phase of the experiment was initiated: two mice were placed in the training chamber comprised of a clear Plexiglas chamber with ample food for 20 minutes for the first day, whereas one mouse was placed in the same chamber on day 2. The training chamber has a single slit through which the mouse can retrieve a pellet of food placed on the outside right in front of the slit. For days 3-7 of the shaping phase, mice were placed individually in the training chamber. Mice show a preference to use one forelimb during this phase (right or left) and subsequent training is being performed such that it allows pellet reaching with the forelimb of preference for each subject (“dominant” forelimb). During the training period, mice were placed in a two-slit chamber individually and food pellet was placed only on the slit corresponding to the dominant forelimb of each mouse. Retrieval attempts were recorded and characterized as successful only if a mouse retrieved the pellet and put it in its mouth. Any other attempt was defined as “failure”. The training was continued for 7 days, and by that time the mouse performance leveled off. Following completion of training, mice were injured with impact-acceleration (60 g×1 m) and their performance in the task was tested daily for 14 days as described above. For SARM1 KO and WT mice, no additional surgical manipulation was performed apart from the injury. For FK866-treated and vehicle-treated groups, immediately after injury or at a specified time after injury, we implanted an osmotic pump continuously delivering drug or vehicle. The average performance of mice over the 14-day period was used in statistical calculations for determining group size effect, using Student's t-test in GraphPad Prism 4.

### Example XI

This example demonstrates nicotinamide phosphoribosyltransferase (NAMPT) inhibition as a therapeutic strategy for Wallerian degeneration.

Wallerian degeneration (WD) is an evolutionary conserved and stereotyped axon self-destruction program that is activated in mechanically and/or metabolically compromised axons and as such contributes to the pathology observed in a spectrum of neurological diseases, including traumatic brain injury and peripheral neuropathies and possibly neurodegenerative syndromes. An instructive signal of WD seems to be the axonal depletion of the NAD+ synthesizing enzyme NMNAT2, the increase of its substrate NMN and the subsequent activation of the NAD+ degrading sterile a and Toll/interleukin-1 receptor (TIR) motif containing 1 (SARM1) protein.

In the absence of available SARM1 inhibitors, experiments described herein established the inhibition of the NMN synthesizing enzyme NAMPT, as an alternative therapeutic strategy in our in vitro axotomy models. Wild type or SARM1 KO embryonic mouse dorsal root ganglion (DRG) cells were cultured in poly-dimethylsiloxane microfluidic devises for axon compartmentalization. Axons were cut with a razor blade and treated with the NAMPT inhibitor FK866 or vehicle at different time points. Images of axons were taken at different intervals and the degeneration index was calculated. Maximum protection against WD was observed with treatment at 2 hours post-injury which was similar to the protection afforded by SARM1 deletion. Importantly treatment after the onset of visible fragmentation was still effective in delaying further fragmentation. A group of other available NAMPT inhibitors (CHS-828, GPP78, STF118804, STF31) was also tested at different concentrations and showed comparably strong efficacy. Axonal preparations at different time points following axotomy were also used for metabolomic profiling and western blot assessment of injury related signals. Such experiments indicated that NAMPT inhibition by a variety of small molecules—some of which have already been tested in clinical trials—is an attractive therapeutic strategy against WD. Most importantly such experiments demonstrated that the molecular decision for WD is stochastic and temporally separated from the time of injury, effectively allowing for a clinically-relevant therapeutic window of intervention.

Experiments with Microfluidic Based Axon Degeneration Assay

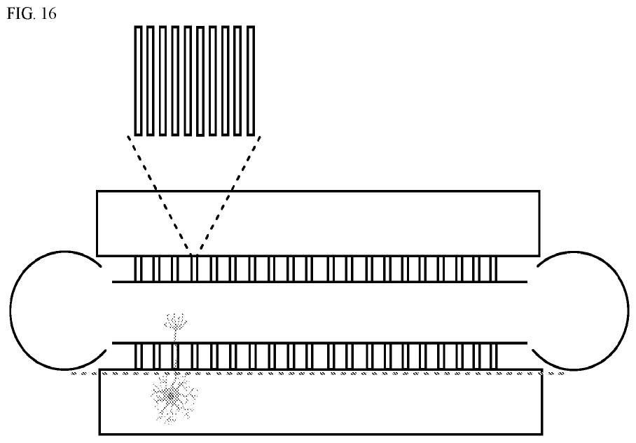

A poly-dimethylsiloxane (PDMS)-based microfluidic device described by Hosmane and colleagues was used for these experiments (see, FIG. 16) (see, Hosmane, et al., Lab Chip 2010, 10, 741-747). This device provisions a perikaryal compartment where nerve cells are first placed and a juxtaposed array of 200 microchannels towards which axons grow and through which they traverse to reach a third axonal compartment. Mouse dorsal root ganglion (DRG) cells were prepared from E12-13 embryos and plated to the perikaryal compartment. After 6-7 days of culture at 37° C. and 5% CO2, axons were cut using a razor blade with vehicle or reagents. Images of the axonal compartment were taken at various time intervals at 20× magnification. Images were then analyzed for degeneration index (DI) which indicates percentage of fragmented axons over total axonal area, and drug-treated axons were compared to vehicle treated or uninjured axons. Live time-lapse images were taken as needed.

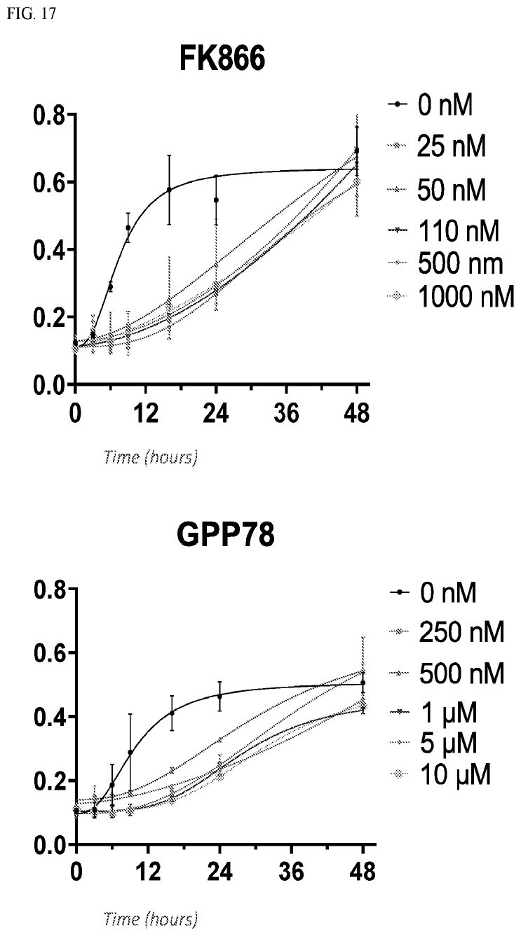

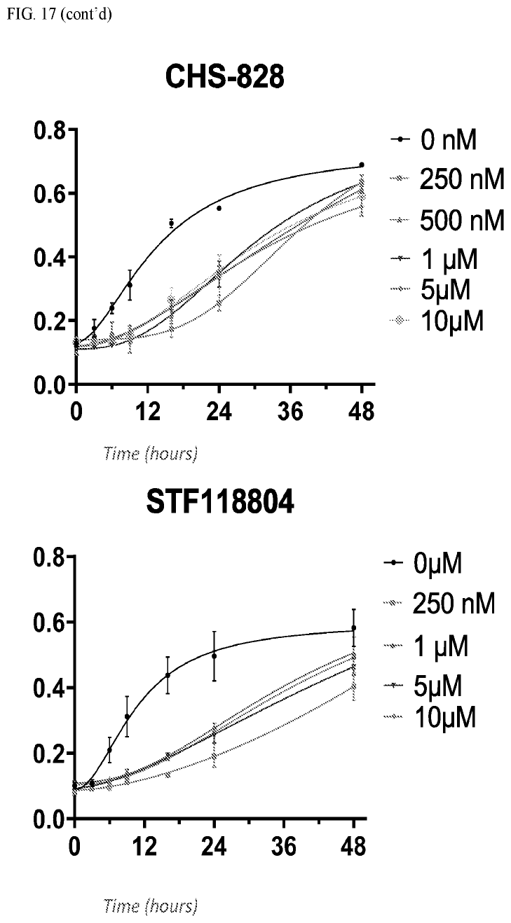

NAMPT inhibitors FK866, STF118804, CHS-828, GPP78, were tested at the indicated doses 2 hours following axotomy and dose responsive curves determined (see, Table I and FIG. 17).

TABLE I
Dose response curves for NAMPT inhibitors

| | | | |
|---|---|---|---|
| | | Range of | Optimized |
| | CAS | Concentrations | Concentration |
| Compound | No | tested | used |

NAMPT inhibitors

| | | | | | |
|---|---|---|---|---|---|
| CHS-828 | 200484-11-3 | 250 | nM-10 μM | 250 | nM |
| FK866 | 658084-64-1 | 25 | nM-1 μM | 100 | nM |
| GPP78 | 1202580-59-3 | 250 | nM-10 μM | 1 | μM |
| STF118804 | 894187-61-2 | 250 | nM-10 μM | 250 | nM |
| STF31 | 724741-75-7 | 250 | nM-10 μM | 2 | μM |

MAP Kinase inhibitors

| | | | | |
|---|---|---|---|---|
| GNE3511 | 1496581-76-0 | — | 500 | nM |
| Tozasertib | 639089-54-6 | — | 1 | μM |
| Sunitinib | 341031-54-7 | — | 500 | nM |

Other

| | | | | |
|---|---|---|---|---|
| NMN | 1094-61-7 | — | 500 | μM |

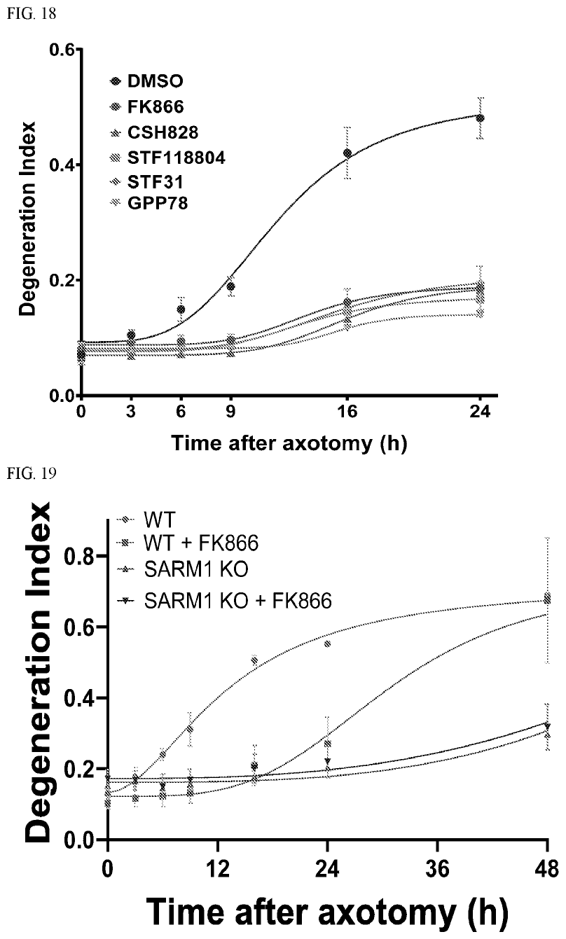

A head to head comparison of different NAMPT inhibitors was conducted wherein NAMPT inhibitors were tested 2 hours following axotomy with the optimized doses (see FIG. 18).

Wild type and SARM1 knock-out (KO) DRGs were plated in microfluidic devices and subjected to axotomy, in the presence or absence of FK866. FIG. 19 demonstrates that NAMPT inhibition was shown to afford similar protection to axons as SARM1 deletion up to 24 hours.

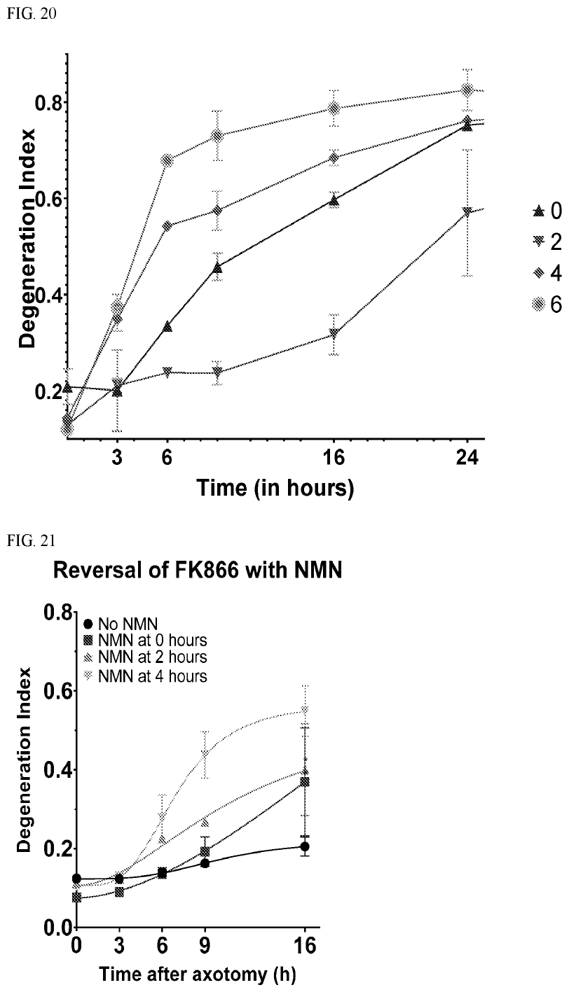

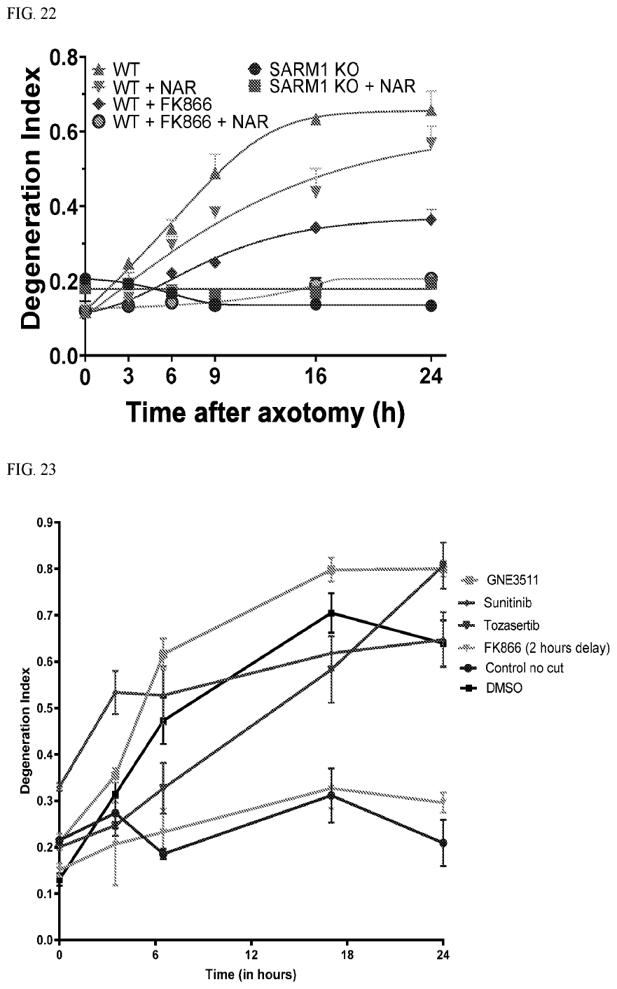

Axotomized axons were treated with the NAMPT inhibitor FK866 at the time of injury or with delayed schedules as indicated (at 0,2,4,6 hours following injury) demonstrating an optimal therapeutic window of treatment onset within two to six hours of injury (FIG. 20). Such optimal effects of NAMPT inhibition were shown to be reversed with NMN (FIG. 21). Axotomized axons were treated with the NAMPT inhibitor FK866 and/or the NAD+ precursor metabolite nicotinamide riboside (NAR) at the time of injury. Addition of NAR further augmented the protective effect of FK866. DRGs were treated with the MAPK inhibitors (Sunitinib, Tozasertib or GNE3511) 8 hours before injury. Treatment effect was compared to control (DMSO) and delayed NAMPT inhibition (FIG. 23).

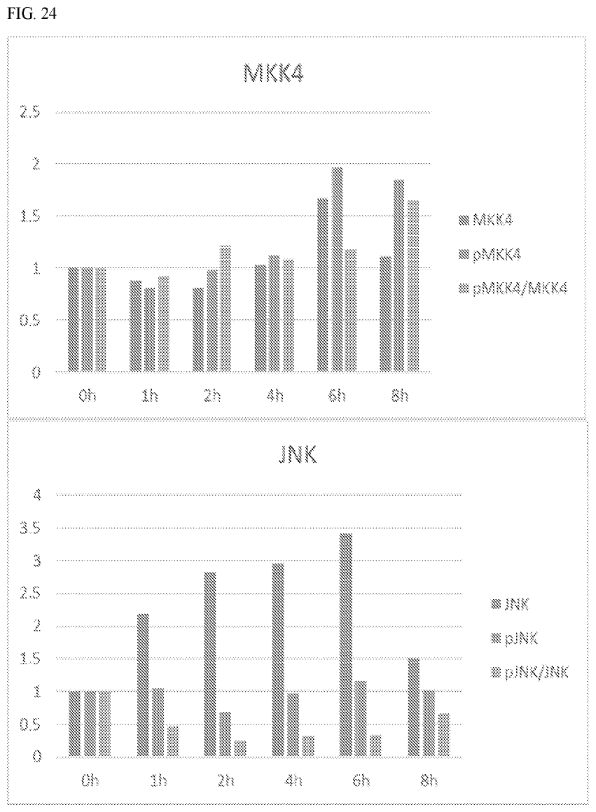

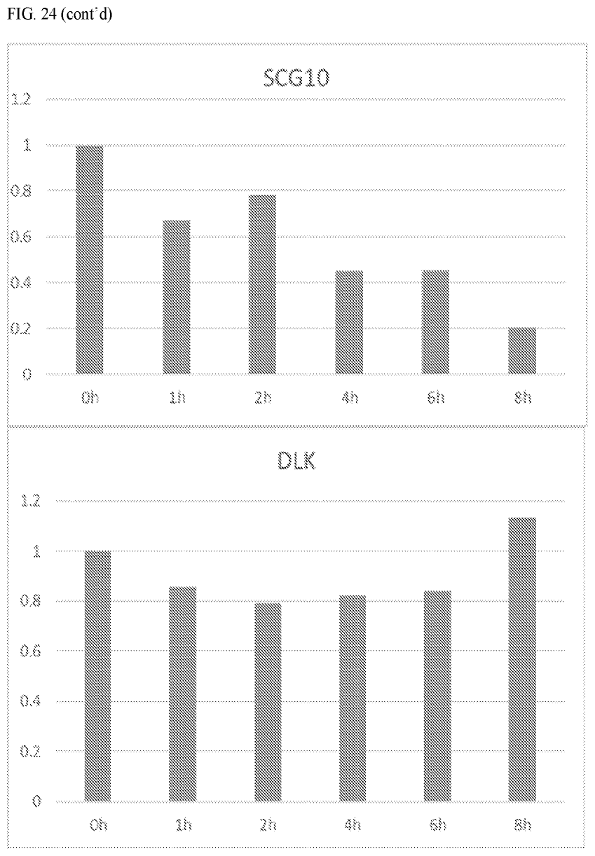

Biochemical Investigation of Axon Degeneration Using Western Blots 250000 neurons were plated as drops in 6-well plates, which allowed the separation and sampling of cell and axon only fractions after 2-3 weeks of culture. After axotomy with a blade, cell bodies were removed and axons were lysed in SDS-based lysis buffer for further SDS-PAGE and immunoblot analysis with total protein normalization. FIG. 24 demonstrates protein changes for MKK4, JNK, SCG10, and DLK following axotomy in wild type, naïve axon fractions as a function of time.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating or preventing a neuropathy or axonopathy in a mammal in need thereof, the method comprising administering to the mammal an effective amount of an agent that acts by decreasing SARM1 and/or NAMPT activity and/or expression in diseased and/or injured neurons and supporting cells, wherein the agent is a small molecule, a nucleic acid, or an antibody.

2. The method of claim 1, wherein the agent is selected from FK866, CHS-828, GPP78, STF118804, STF31, and chemically related or unrelated molecules that inhibit NAMPT.

3. (canceled)

4. The method of claim 1, wherein the neuropathy or axonopathy is hereditary or congenital or associated with neurodegenerative disease, motor neuron disease, neoplasia, endocrine disorder, metabolic disease, nutritional deficiency, atherosclerosis, an autoimmune disease, mechanical injury, chemical or drug-induced injury, thermal injury, radiation injury, nerve compression, retinal or optic nerve disorder, mitochondrial dysfunction, progressive dementia demyelinating diseases ischemia and/or stroke infectious disease; or inflammatory disease.

5. The method of claim 4, wherein the neuropathy or axonopathy is caused by a traumatic brain injury, wherein the onset of treating is within one to seven hours of injury, wherein the duration of treating is between three to fourteen days.

6. The method of claim 1, wherein the mammal is a human.

7-10. (canceled)

11. A method for reducing axonal degradation in a subject with or at risk for developing axonal degradation, the method comprising: selecting a subject with or at risk for developing axonal degradation; and treating the subject with an effective amount of a composition that inhibits SARM1 and/or NAMPT activity and/or expression for a time sufficient to inhibit SARM1 activity and/or expression, thereby reducing axonal degradation in the subject, wherein the composition that inhibits SARM1 and/or NAMPT activity and/or expression comprises a small molecule, a nucleic acid, or an antibody.

12. The method of claim 11, wherein the subject has or is at risk of one or more of the following:
a neurodegenerative disease;
a traumatic brain injury; and
diabetes with or without diabetic neuropathy.

13. The method of claim 11, wherein the axonal degradation is in the central nervous system (CNS) and/or the peripheral nervous system (PNS).

14-16. (canceled)

17. The method of claim 11, wherein the composition that inhibits SARM1 activity and/or expression comprises an agent capable of increasing nicotinamide levels.

18. The method of claim 17, wherein the agent is FK866.

19. (canceled)

20. The method of claim 11, wherein the composition that inhibits NAMPT activity and/or expression is selected from CHS-828, GPP78, STF118804, and STF31.

21. The method of claim 11, wherein the subject has developed axonal degradation, wherein the onset of treating is within two to six hours of axonal degradation development, wherein the duration of treating is between three to fourteen days.

22. The method of claim 5, wherein the onset of treating is within 3-5 hours, 2-5 hours, 2-6 hours, 2-7 hours, 2.5 hours to 6.5 hours, or 1.5 to 6.5 hours.

23. The method of claim 21, wherein the onset of treating is within 3-5 hours, 2-5 hours, 2-6 hours, 2-7 hours, 2.5 hours to 6.5 hours, or 1.5 to 6.5 hours.

24. A method of treating or preventing a neuropathy or axonopathy in a mammal in need thereof, the method comprising administering to the mammal an effective amount of a composition comprising 1) an agent that acts by decreasing SARM1 and/or NAMPT activity and/or expression in diseased and/or injured neurons and supporting cells, and 2) nicotinamide riboside (NAR), wherein the agent is a small molecule, a nucleic acid, or an antibody.

25. The method of claim 24, wherein the agent is selected from FK866, CHS-828, GPP78, STF118804, STF31, and a chemically related or unrelated molecule that inhibit NAMPT.

26. (canceled)

27. The method of claim 24, wherein the neuropathy or axonopathy is hereditary or congenital or associated with neurodegenerative disease, motor neuron disease, neoplasia, endocrine disorder, metabolic disease, nutritional deficiency, atherosclerosis, an autoimmune disease, mechanical injury, chemical or drug-induced injury, thermal injury, radiation injury, nerve compression, retinal or optic nerve disorder, mitochondrial dysfunction, progressive dementia demyelinating diseases ischemia and/or stroke infectious disease; or inflammatory disease.

28. The method of claim 27, wherein the neuropathy or axonopathy is caused by a traumatic brain injury, wherein the onset of treating is within one to seven hours of injury, wherein the duration of treating is between three to fourteen days.

29. The method of claim 24, wherein the mammal is a human.

30. The method of claim 24, wherein the onset of treating is within 3-5 hours, 2-5 hours, 2-6 hours, 2-7 hours, 2.5 hours to 6.5 hours, or 1.5 to 6.5 hours.

31-32. (canceled)
